SEQUENCE LISTING (8 sequences)

SEQ ID NO: 1 - DNA, 22 bp, Artificial
  N-FLAG_forward primer
ctacaaagacgatgacgacaag

SEQ ID NO: 2 - DNA, 23 bp, Artificial
  N-MYC_forward primer
gaagaggatctgggaggttcagg

SEQ ID NO: 3 - DNA, 19 bp, Artificial
  N-tag_reverse primer
cttcttcctggcttcctcg

SEQ ID NO: 4 - DNA, 23 bp, Artificial
  C-tag_forward primer
cccactgtctctctacaatgagg

SEQ ID NO: 5 - DNA, 22 bp, Artificial
  C-FLAG_reverse primer
gtcgtcatcgtctttgtagtcg

SEQ ID NO: 6 - DNA, 21 bp, Artificial
  C_MYC_reverse primer
gtttttgttcgctgcctcctg

SEQ ID NO: 7 - DNA, 20 bp, Artificial
  Actin_forward primer
caaccttccttcctgggcat

SEQ ID NO: 8 - DNA, 20 bp, Artificial
  Actin-reverse primer
gattttcatcgtgctgggcg

(19) United States
(12) Patent Application Publication
(10) Pub. No.: US 2023/0183805 A1
(43) Pub. Date: Jun. 15, 2023

(54) RESIDUAL ENZYME ACTIVITY IN METABOLIC DISEASE

(71) Applicant: UNIVERSITÄT HEIDELBERG, Heidelberg (DE)

(72) Inventors: Matthias Zielonka, Heidelberg (DE); Roland Posset, Heidelberg (DE); Jürgen Okun, Heidelberg (DE); Stefan Kölker, Heidelberg (DE)

(21) Appl. No.: 17/607,436

(22) PCT Filed: Apr. 30, 2020
(86) PCT No.: PCT/EP2020/062085

§ 371 Date: Oct. 29, 2021

(30) Foreign Application Priority Data
May 2, 2019 (EP) 19172331.1

(51) Int. Cl. C12Q 1/6883; C12Q 1/6869

(52) U.S. Cl. CPC C12Q 1/6883; C12Q 1/6869; C12Q 2600/156; C12Q 2600/16

(57) ABSTRACT

Described herein is a method for identifying a subject suffering from a severe form of a metabolic disease, including a) obtaining (i) at least partial nucleic acid sequences of at least two alleles of a gene contributing to the metabolic disease or at least partial amino acid sequences of the polypeptides expressed therefrom; and (ii) a residual activity of the polypeptides expressed from the at least two alleles, and b) based on the result of step a), identifying a subject suffering from a severe form of a metabolic disease. Also described herein are methods, data collections, devices, kit, and computer program products related thereto.

### CROSS-REFERENCE TO RELATED APPLICATIONS

This application is a U.S. National Phase Application of International Patent Application No. PCT/EP2020/062085, filed Apr. 30, 2020, which claims priority to European Patent Application No. 19172331.1, filed May 2, 2019, the entire contents of which are hereby incorporated by reference herein.

### REFERENCE TO AN ELECTRONIC SEQUENCE LISTING

The contents of the electronic sequence listing (UH15830PC_Sequence listing.txt; Size: 1,704 bytes; and Date of Creation: Oct. 18, 2021) is herein incorporated by reference in its entirety.

The present invention relates to a method for identifying a subject suffering from a severe form of a metabolic disease, comprising a) obtaining (i) at least partial nucleic acid sequences of at least two alleles of a gene contributing to said metabolic disease or at least partial amino acid sequences of the polypeptides expressed therefrom; and (ii) a residual activity of the polypeptides expressed from said at least two alleles, and b) based on the result of step a), identifying a subject suffering from a severe form of a metabolic disease; and to methods, data collections, devices, kit, and computer program products relates thereto.

Citrullinemia type 1 (CTLN1) is a rare urea cycle disorder caused by autosomal recessively inherited deficiency of the cytosolic enzyme argininosuccinate synthetase type 1 (ASS1-D; MIM #215700) due to genetic variations in the ASS1 gene located on 9q34.11. CTLN1 is the second to third most common urea cycle disorder (UCD) with an estimated overall incidence of 1 in 250,000 individuals (Summar et al. (2013), Mol Genet Metab 110(1-2):179-80; Posset et al. (2019), J Inherit Metab Dis. 42(1):93-106.). The clinical presentation of CTLN1 covers a wide range including symptomatic individuals with severe and life-threatening hyperammonemic events (HAEs) within the first 28 days of life (early onset, EO) or with a more variable and mild to moderate phenotype of individuals presenting after the newborn period (late onset, LO). In addition, some individuals with CTLN1 remained asymptomatic without specific therapy. These are the major but rough cornerstones of current clinical classification (EO, LO, asymptomatic) in individuals with CTLN1 (Kolker et al. (2015), J Inherit Metab Dis.; 38(6):1041-57; Kolker et al. (2015) J Inherit Metab Dis. 38(6):1059-74.). A meta-analysis recently demonstrated that neonatal mortality in individuals with CTLN1 with EO is still high and has not significantly improved for more than three decades (Burgard et al. (2016), J Inherit Metab Dis. 39(2):219-29), despite implementation of pharmacologic and extracorporeal detoxification for emergency treatment of HAEs, and low protein diet and nitrogen scavengers for maintenance treatment. Liver transplantation (LTx) seems a better option than conventional therapy to alter the disease course or to improve the neurocognitive outcome of individuals who survived the initial HAE or were diagnosed while still being asymptomatic (Posset et al. (2019) Ann Neurol. doi: 10.1002/ana.25492, PMID: 31018246).

Large observational registry studies for UCDs in North America and Europe have identified clinical, such as early disease onset, or biochemical variables, such as high initial peak plasma ammonium concentration (NH₄⁺ₘₐₓ), to be correlated with a poor long-term outcome in UCDs (Posset et al. (2016), J Inherit Metab Dis. 39(5):661-72; Waisbren et al. (2018) J Inherit Metab Dis. 41(4):657-67). Based on a systematic data collection, currently more than 100 disease-causing ASS1 genetic variants are known (Diez-Fernandez et al. (2017), Hum Mutat. 38(5):471-84.), however the impact of the genotype on the phenotypic presentation remains insufficiently understood for individuals with CTLN1.

There is, thus, a need in the art to provide reliable means to identify or predict severe metabolic disease such as CTLN1 in a subject. In particular, there is a need to provide means and methods avoiding at least in part the drawbacks of the prior art as discussed above.

This problem is solved by the means and methods with the features of the independent claims. Preferred embodiments, which might be realized in an isolated fashion or in any arbitrary combination are listed in the dependent claims.

Accordingly, the present invention relates to a method for identifying a subject suffering from a severe form of a metabolic disease, comprising

a) obtaining (i) at least partial nucleic acid sequences of at least two alleles of a gene contributing to said metabolic disease or at least partial amino acid sequences of the polypeptides expressed therefrom; and (ii) a residual activity of the polypeptides expressed from said at least two alleles, and

b) based on the result of step a), identifying a subject suffering from a severe form of a metabolic disease.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value±20%, more preferably +10%, most preferably ±5%. Further, the term “essentially” indicates that deviations having influence on the indicated result or use are absent, i.e. potential deviations do not cause the indicated result to deviate by more than +20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1%, most preferably less than 0.1% by weight of non-specified component(s). In the context of nucleic acid sequences, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis.

The term “polynucleotide”, as used herein, refers to a linear or circular nucleic acid molecule. The polynucleotide of the present invention shall be provided, preferably, either as an isolated polynucleotide (i.e. isolated from its natural context) or in genetically modified form. The term encompasses single as well as double stranded polynucleotides. Moreover, comprised are also chemically modified polynucleotides including naturally occurring modified polynucleotides such as glycosylated or methylated polynucleotides or artificially modified derivatives such as biotinylated polynucleotides. The polynucleotide of the present disclosure encodes a polypeptide contributing to metabolic disease; thus, preferably, the polynucleotide comprises at least one open reading frame (ORF) of an allele, preferably encodes one ORF of an allele of a gene contributing to metabolic disease. Preferably, the gene contributing to metabolic disease is modified by a deletion, addition and/or substitution of at least one nucleotide leading to a truncation, disruption, or mutation of the polypeptide produced from the said gene, as compared to the unmutated gene. Such modifications also encompass point mutations resulting in an exchange of at least one amino acid for a different amino acid, resulting in the generation of a stop codon, as well as modifications resulting in a shift of the open reading frame. The polypeptides being produced from such modified polynucleotides preferably comprise one or more amino acid exchanges, are abnormally short or abnormally long and have at least reduced biological function, preferably at least reduced enzymatic activity. Preferred are point mutations in a gene which result in a polypeptide with no or with a decreased biological function. Also preferably, a deletion, addition and/or substitution of at least one nucleotide can be, preferably, present which leads to an inactivation of the transcriptional control sequence (i.e. the promoter) which governs expression of the gene. Moreover, sequences may be, preferably, introduced which in the transcribed RNA result in increased RNA degradation. The polynucleotides of the present invention either essentially consist of the aforementioned nucleic acid sequences or comprise the aforementioned nucleic acid sequences. Thus, they may contain further nucleic acid sequences as well. Specifically, the polynucleotides of the present invention may encode fusion proteins wherein one partner of the fusion protein is a polypeptide being encoded by a nucleic acid sequence recited above. Such fusion proteins may comprise as additional part polypeptides for monitoring expression (e.g., green, yellow, blue or red fluorescent proteins, alkaline phosphatase and the like) or so called “tags” which may serve as a detectable marker or as an auxiliary measure for purification purposes. Tags for the different purposes are well known in the art and comprise FLAG-tags, MYC-tags, 6-histidine-tags, and the like.

The term “polypeptide” is in principle understood by the skilled person. The term “polypeptide contributing to metabolic disease”, as used herein, relates to a polypeptide the activity of which, when absent or produced at a reduced amount in a subject, contributes, preferably causes, most preferably is the only cause of metabolic disease as specified herein below. Thus, preferably, the polypeptide contributing to metabolic disease is a mutein of an enzyme normally present in a subject, the activity of which is required to maintain normal metabolism. Preferred polypeptides contributing to metabolic disease are specified elsewhere herein.

The method for identifying a subject of the present invention, preferably, is an in vitro method. Moreover, it may comprise steps in addition to those explicitly mentioned above. For example, further steps may relate, e.g., to providing a sample for step a), or performing additional evaluation steps, in particular additional diagnostic steps, before or after step b). Moreover, one or more of said steps may be performed by automated equipment.

As used herein, the term “identifying” refers to assessing whether a subject suffers from a severe form of a metabolic disease. As will be understood by those skilled in the art, such an assessment, although preferred to be, may usually not be correct for 100% of the investigated subjects. The term, however, requires that, preferably, a statistically significant portion of subjects can be correctly assessed. Whether a portion is statistically significant can be determined without further ado by the person skilled in the art using various well known statistic evaluation tools, e.g., determination of confidence intervals, p-value determination, Student's t-test, Mann-Whitney test, etc.. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-value is, preferably, 0.05, more preferably, is 0.01, most preferably, is 0.001. Preferably, identifying a subject suffering from a severe form of a metabolic disease is providing an indication that said subject is suffering from a severe form of a metabolic disease. As is understood by the skilled person, said identifying may comprise further evaluation steps as regards the severity of disease, e.g., preferably, obtaining anamnesis data, performing general physical and/or mental examination, obtaining additional metabolic data, and the like. Thus, preferably, identifying is not diagnosing. It is, however, also envisaged that identifying preferably is diagnosing a severe form of a metabolic disease in a subject. As the skilled person understands, in a preferred embodiment, identifying is differentiating between a severe and a mild form of a metabolic disease, preferably wherein a “mild” metabolic disease is a non-severe metabolic disease. In a further preferred embodiment, identifying comprises stratifying subjects into groups of different severity of metabolic disease, e.g. severe or mild metabolic disease; or severe, intermediate, or mild metabolic disease. In a preferred embodiment, the treatment of said different groups of different severity of metabolic disease is non-identical; thus, in a preferred embodiment, the method for identifying is an aid for the medical practitioner for taking a treatment decision.

The term “subject” relates to a metazoan organism, preferably an animal, more preferably a mammal, most preferably a human being. Preferably, the subject is known or suspected to suffer from metabolic disease.

The term “host cell”, as used herein, relates to any cell capable of expressing a gene contributing to metabolic disease. Thus, the host cell preferably is an archeal, a bacterial, or a eukaryotic cell. More preferably, the cell is a metazoan cell, still more preferably a mammalian cell, most preferably a human cell. Preferably, the host cell is a standard cultured mammalian cell, more preferably is a COS cell, most preferably a COS-7 cell. Preferably, the host cell does not express the gene contributing to metabolic disease of interest or expresses the gene contributing to metabolic disease only to a reduced extent.

The term “metabolic disease” is understood by the skilled person to relate to any type of disease disrupting and/or caused by a disruption of normal metabolism in a subject. Preferably, as used herein, the term relates to a disease caused by a non-normal, i.e. increased or decreased, enzymatic activity of one type of polypeptide in body cells of a subject. Preferably, metabolic disease is caused by an enzymatic activity which is diminished relative to the enzymatic activity in corresponding body cells of a subject not affected by said metabolic disease. Thus, preferably, the metabolic disease is caused by a reduced or abolished activity of one type of enzyme in a subject. Preferably, the metabolic disease is an inherited or inheritable disease, i.e. preferably, caused by at least one mutation in a gene encoding said enzyme; also preferably, the metabolic disease is a monogenic metabolic disease. Also preferably, the metabolic disease is a recessive metabolic disease. Also preferably, the metabolic disease is an autosomal metabolic disease, more preferably an autosomal recessive metabolic disease, most preferably a monogenic autosomal recessive metabolic disease. Preferably, the metabolic disease is Citrullinemia type 1 (CTLN1) or Argininosuccinate Lyase deficiency (ASL-D). Thus, preferably, the metabolic disease is Citrullinemia type 1 (CTLN1), wherein, more preferably, the gene contributing to said metabolic disease is a mutated form of the argininosuccinate synthetase 1 (ASS1) gene, even more preferably the human ASS1 gene, most preferably the gene with Genbank Acc No: NG_011542.1. Also preferably, the metabolic disease is Argininosuccinate Lyase deficiency (ASL-D), wherein, more preferably, the gene contributing to said metabolic disease is a mutated form of the argininosuccinate lyase (ASL) gene, more preferably the human ASL gene, even more preferably the gene with Genbank Acc No.: AF376770.1 In a preferred embodiment, the metabolic disease is caused by a reduced activity of ornithine transcarbamylase (OTC, (EC 2.1.3.3)). Thus, in a preferred embodiment, the metabolic disease is OTC deficiency, wherein, more preferably, the gene contributing to said metabolic disease is a mutated form of the OTC gene, even more preferably the human OTC gene, most preferably the gene with Genbank Acc No: NG_008471.1.

The term “severe form of a metabolic disease”, as used herein, relates to a form of a metabolic disease in which the residual activity of the enzyme contributing to the metabolic disease as specified herein above is less than 25%, preferably less than 15%, more preferably less than 10%, even more preferably less than about 8%, most preferably less than 5%. Thus, as used herein, the term “severe form of a metabolic disease” preferably relates to the severity of metabolic distortion and only indirectly to the severity of symptoms caused thereby; it will, however, be understood by the skilled person that increasing distortion of metabolism will generally correlate with the severity of symptoms caused by the distortion. Preferably, residual activity is determined as specified elsewhere herein, more preferably as described herein in the Examples. In case the metabolic disease is Citrullinemia type 1, preferred symptoms of severe disease are selected from the list consisting of recurrent hyperammonemic decompensations, coma, seizures, sepsis-like appearance, severe cognitive impairment, movement disorders and hepatic dysfunction. In case the metabolic disease is Argininosuccinate Lyase deficiency, preferred symptoms of severe disease are selected from the list consisting of, in analogy to Citrullinemia type 1, recurrent hyperammonemic decompensations, coma, seizures, sepsis-like appearance, severe cognitive impairment and progressive hepatic dysfunction. In a preferred embodiment, a subject identified to suffer from severe metabolic disease is treated for said metabolic disease, preferably with treatments known to the skilled person and described herein above. In a preferred embodiment, a subject not identified to suffer from severe metabolic disease is not treated or receives a different treatment. In a preferred embodiment, in view of the above, a severe form of a metabolic disease may also be further subdivided into different degrees of severity, e.g. cases with residual activity of the enzyme contributing to the metabolic disease of less than 25% to 10%, and cases with residual activity of the enzyme contributing to the metabolic disease of less than 10%.

As used herein, the term “gene contributing to a metabolic disease” relates to a gene encoding an enzyme contributing to the metabolic disease as specified herein above; thus, a gene contributing to a metabolic disease is a gene which, when mutated, preferably encodes a polypeptide having a reduced enzymatic activity compared to the non-mutated gene. In accordance, the term “gene contributing to metabolic disease” is used herein for a gene, including all, preferably naturally occurring, alleles, whether they actually cause metabolic disease or not. Preferred genes and alleles contributing to metabolic disease are known in the art; more preferred genes and alleles contributing to metabolic disease are referred to herein above. As will be understood by the skilled person, a diploid cell, i.e. a normal, non-reproductive cell, of a subject will contain two, preferably non-identical, alleles of all autosomal genes. Thus, a subject preferably has two non-identical copies of a gene contributing to metabolic disease; preferably, at least one, more preferably both, of said copies (alleles) comprise at least one mutation contributing to a decrease or loss of enzymatic activity; as is understood from the above, said two alleles preferably comprise a non-identical mutation or mutations. In accordance, in case the subject is a diploid organism, preferably a mammal, the term “at least two alleles” preferably relates to “two alleles” The term “at least partial nucleic acid sequence” includes any sequence information on or derivable from a gene contributing to a metabolic disease, including information on only parts of the nucleic acid sequence. Preferably, said sequence information is characteristic of a mutated form (allele) of a gene contributing to metabolic disease encoding a polypeptide having a decreased enzymatic activity. Thus, preferably, the at least partial nucleic acid sequence comprises at least sequence information on at least one, more preferably at least two, even more preferably at least three, still more preferably at least four, even more preferably at least five, most preferably at least six, nucleotide(s) and its (their) position in the gene sequence or cDNA sequence of the gene contributing to metabolic disease. More preferably, the at least partial nucleic acid sequence comprises at least sequence information on at least seven, more preferably at least eight, still more preferably at least nine, most preferably at least ten contiguous nucleotides of the gene sequence or cDNA sequence of the gene contributing to metabolic disease. Still more preferably, the at least partial nucleic acid sequence comprises at least the sequence of at least one, more preferably at least two, still more preferably at least three, most preferably at least four exons of the gene sequence of the gene contributing to metabolic disease. Still more preferably, the at least partial nucleic acid sequence comprises the sequence of at least 25%, more preferably at least 50%, still more preferably at least 75% even more preferably at least 90% of the cDNA sequence of the gene contributing to metabolic disease. Most preferably, the at least partial nucleic acid sequence comprises the sequence of the open reading frame of the gene encoding metabolic disease, preferably its cDNA sequence.

In accordance, the term “at least partial amino acid sequence” relates to any sequence information on a polypeptide contributing to metabolic disease. Preferably, said sequence information is characteristic of a mutated form having a decreased enzymatic activity. Thus, preferably, the at least partial amino acid sequence comprises at least sequence information on at least one, more preferably at least two, even more preferably at least three, still more preferably at least four, even more preferably at least five, most preferably at least six, amino acid(s) and its (their) position in the amino acid sequence of the polypeptide (enzyme) contributing to metabolic disease. More preferably, the at least partial amino acid sequence comprises at least sequence information on at least seven, more preferably at least eight, still more preferably at least nine, most preferably at least ten contiguous amino acids of the polypeptide (enzyme) contributing to metabolic disease. Still more preferably, the at least partial amino acid sequence comprises at least the sequence of at least one, more preferably at least two, still more preferably at least three, most preferably at least four peptides, in particular proteolytic peptides, of the polypeptide contributing to metabolic disease. As is understood by the skilled person, sequence information on at least a partial amino acid sequence of a polypeptide may also be obtained by determining the mass, m/z ratio, or similar parameters of one or more, preferably proteolytic, peptides of the polypeptide (enzyme) contributing to metabolic disease.

The term “obtaining”, as used herein, relates to acquiring the indicated information, in particular a value of a parameter such as an at least partial sequence or a residual activity, in a manner enabling basing the identification of a subject suffering from a severe form of a metabolic disease on said information. Thus, preferably, obtaining is reading the information from data carrier, e.g. in the form of a data sheet, a sequencing file, a mass spectrum, or the like; or from a database comprising at least the relevant value or values. More preferably, obtaining is determining said information as specified herein below.

As used herein, the term “determining” relates to providing the indicated information, in particular a value of a parameter such as an at least partial sequence or a residual activity, using a sample of a subject or a biological material derived therefrom. Thus, determining at least a partial nucleic acid sequence, preferably, comprises detecting the sequence of bases in a polynucleotide; appropriate methods are known in the art and include in particular nucleic acid sequencing, hybridization, and restriction enzyme digestion. Preferably, determining at least a partial nucleic acid sequence is performed by nucleic acid sequencing, more preferably Sanger (chain termination) sequencing, pyrosequencing, or any other method deemed appropriate. More preferably, determining at least a partial nucleic acid sequence is performed by Sanger sequencing. Determining at least a partial amino acid sequence, preferably, comprises detecting the sequence of amino acids in a polypeptide; appropriate methods are known in the art and include in particular Edman degradation of the polypeptide or of one or more peptides derived therefrom, detection with sequence-specific antibodies, detection of the molecular mass, m/z-value, or a similar parameter of one or more peptide(s) derived from the polypeptide, and similar methods. Preferably, determining at least a partial amino acid sequence is performed by Edman degradation, more preferably of one or more peptides derived from the polypeptide.

As used herein, the term “determining a residual activity” relates to determining the activity, typically the enzymatic activity, of the polypeptides expressed from the at least two alleles present in a subject. Thus, as used herein, determining a residual activity relates to simultaneous determination of the activity of the polypeptides expressed from the at least two alleles present in a subject. Thus, preferably, determining a residual activity results in the determination of a single value of a residual activity. As will be understood by the skilled person, the residual activity may be expressed as any measure deemed appropriate by the skilled person, e.g. preferably as an activity measured in the assay, as a specific activity, as a relative activity, e.g. compared to normal, as a relative specific activity, and the like. More preferably, the residual activity is expressed as a relative specific activity compared to the specific activity determined with one or two non-mutated alleles.

Preferably, determining a residual activity comprises (i) cloning the sequences of at least two alleles of step a) into expression constructs. The term “expression construct”, as used herein, relates to a polynucleotide encoding a polypeptide contributing to metabolic disease as specified herein, operatively linked to expression control sequences allowing expression in a host cell or isolated fractions thereof. Expression of said polynucleotide comprises transcription of the polynucleotide, preferably into a translatable mRNA. Regulatory elements ensuring expression in eukaryotic cells, preferably mammalian cells, are well known in the art. They, preferably, comprise regulatory sequences ensuring initiation of transcription and, optionally, poly-A signals ensuring termination of transcription and stabilization of the transcript. Additional regulatory elements may include transcriptional as well as translational enhancers. Preferably, the regulatory elements are those of the gene contributing to metabolic disease; more preferably, the regulatory elements are heterologous regulatory elements. E.g., preferably, regulatory elements permitting expression in prokaryotic host cells comprise, e.g., the lac, trp or tac promoter in E. coli, and examples for regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Moreover, inducible expression control sequences may be used in an expression construct encompassed by the present invention. Such inducible expression constructs may comprise tet or lac operator sequences or sequences inducible by heat shock or other environmental factors. Suitable expression control sequences are well known in the art. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art such as Okayama-Berg cDNA expression vector pcDV1 (Pharmacia), pBluescript (Stratagene), pCDM8, pRc/CMV, pcDNA, pcDNA3 (InVitrogene) or pSPORT1 (GIBCO BRL). Preferably, the expression vector is pcDNA5. Preferably, the expression control sequences are identical for all alleles under investigation, more preferably including negative and positive control expression constructs. Expression constructs derived from viruses such as retroviruses, vaccinia virus, adeno-associated virus, herpes viruses, or bovine papilloma virus, may be used for delivery of polynucleotides into host cells. More preferably, expression constructs are transferred into host cells by transfection, preferably by one of the methods known in the art. As is understood by the skilled person, the step of “cloning the sequences of at least two alleles into expression constructs” may be accomplished by any method deemed appropriate by the skilled person; this includes in particular cloning of a chemically synthesized polynucleotide into an expression construct, cloning of a cDNA into expression a construct, exchanging a sub-ORF fragment in an expression construct for a fragment comprising one or more mutations of interest, and mutagenizing an expression construct to introduce into said expression construct one or more mutations of interest, or a combination of the aforesaid methods. Also, for two or more alleles, the aforesaid methods may be independently selected, e.g., preferably, one allele may be cloned as a cDNA, and the other may be cloned by mutagenesis of a pre-existing expression construct. As used herein, the term “expression construct” includes any polynucleotide causing production of a polypeptide contributing to metabolic disease when introduced into a host cell; thus, an expression construct as referred to herein may also be an mRNA.

Preferably, determining a residual activity comprises (ii) expressing said expression constructs, preferably in a suitable host cell. Preferably, said expressing said expression constructs comprises introduction of said expression constructs into a suitable host cell, transcription of the genes contributing to metabolic disease into mRNAs, and translation of said mRNAs into polypeptides by protein biosynthesis. As the skilled person will understand, alleles of interest may also be expressed by transfecting mRNAs encoding said alleles into a host cell, by infection of a host cell with a virus causing expression of said allele, and other methods known to the skilled person. Moreover, expressing the expression constructs may, preferably, be performed in an in vitro translation system or in an in vitro transcription/translation system. In case the two alleles of a diploid subject are expressed, preferably similar amounts of expression construct are transferred into the host cells. Thus, the ratio of first expression construct (encoding a first allele) to second expression construct (encoding a second allele) transferred into host cells preferably is of from 0.7 to 1.3, more preferably of from 0.8 to 1.2, even more preferably of from 0.9 to 1.1. More preferably, essentially equal amounts of expression constructs are transferred into the host cells. Thus, still more preferably, the ratio of first expression construct to second expression construct transferred into host cells is about 1, most preferably is 1.

Preferably, determining a residual activity comprises (iii) determining the residual activity in an enzymatic assay. Methods for determining the activity, in particular the enzymatic activity, of a polypeptide of interest are known in the art and include in particular those referred to herein in the Examples. As used herein, determining the residual activity relates to determining the residual activity of all polypeptides contributing to metabolic disease in one, i.e. in the same, assay mixture. Thus, as used herein, the term preferably does not include separate determination of activities of polypeptides encoded by single alleles of a gene contributing to metabolic disease.

Preferably, the method for identifying a subject suffering from a severe form of a metabolic disease further comprises performing control determinations, e.g. preferably using at least one expression construct not encoding a polypeptide contributing to metabolic disease (negative control), and/or at least one expression construct encoding a known non-mutated polypeptide contributing to metabolic disease (positive control and/or reference). Furthermore, a transfection control encoding an unrelated polypeptide having a detectable, preferably quantifiable, property may be included.

Advantageously, it was found in the work underlying the present invention that the residual activity of a polypeptide contributing to metabolic disease is beneficially determined in an assay determining the activity of both alleles in the same assay. It was further found that the results of such a determination correlate very well with other clinical and diagnostic findings. Thus, the values obtained by the method can be used as surrogate markers and/or predictors of severity of metabolic disease, i.e. preferably, the values obtained by the method can be used to define groups with different severity of disease, e.g. mild disease vs. severe disease, which can each be treated according to the requirements of the specific group.

The definitions made above apply mutatis mutandis to the following. Additional definitions and explanations made further below also apply for all embodiments described in this specification mutatis mutandis.

The present invention further relates to a method of determining a residual activity of an enzyme expressed from at least two alleles of a gene contributing to metabolic disease, comprising

A) expressing said two alleles in a host cell, and

B) determining the activity of said enzyme in said host cell or an extract thereof.

In a preferred embodiment, the present invention also relates to a method of identifying a compound for treating and/or preventing a metabolic disease, comprising

a) contacting a host cell expressing at least two alleles of a gene contributing to said metabolic disease with a candidate compound,

b) simultaneously determining residual activity of the polypeptides expressed from said at least two alleles in said host cell or an extract thereof,

c) comparing the residual activity determined in step b) to a control activity, and

d) based on comparison step c), identifying a compound for treating and/or preventing said metabolic disease.

The method of identifying a compound, preferably, is an in vitro method. Moreover, the method may comprise further steps, e.g. providing at least one expression construct and/or introducing said at least one expression construct into a host cell. Moreover, one or more steps may be assisted or performed by automated equipment. Also, in the method of identifying a compound, the host cell preferably is a cell of the same species and, preferably the same tissue and/or cell type as is causing the metabolic disease; thus, in such case, the host cell preferably is a mammalian cell, preferably a human cell.

The term “candidate compound” is used herein in a broad sense relating to any chemical compound suspected to be a compound for treating and/or preventing a metabolic disease; thus, the term, preferably, relates to any compound not having previously been tested negative for a metabolic disease treating and/or preventing activity.

The term “control activity” is understood by the skilled person and may relate to a control of the same host cells expressing said alleles of a gene contributing to said metabolic disease, but not contacted with said candidate compound (negative control); in such case, a residual activity which is increased compared to said control is indicative of a compound active in treating and/or preventing a metabolic disease. Preferably, a further control activity may be a positive control, i.e. an activity of at least one, preferably at least two, unmutated alleles of said gene contributing to said metabolic disease; in such case, a residual activity which is closer in value to said positive control compared to the activity in the absence of said candidate compound is indicative of a compound active in treating and/or preventing a metabolic disease.

The present invention also relates to a data collection comprising sequence data of least two alleles of a gene related to a metabolic disease and a value of a residual enzyme activity correlating therewith.

The term “data collection” refers to a collection of data which may be physically and/or logically grouped together. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets, in particular sequence data of least two alleles, being indicative for a medical condition or effect as set forth above (e.g. a query search). Thus, if an identical or similar data set can be identified in the data collection, the test data set will be associated with the said medical condition or effect. More preferably, the data collection comprises residual activity values corresponding to a variety of, preferably all known, combinations of least two alleles of a gene related to a metabolic disease. The term “data storage medium” as used herein encompasses data storage media which are based on single physical entities such as a CD, a CD-ROM, a hard disk, optical storage media, or a diskette. Moreover, the term further includes data storage media consisting of physically separated entities which are operatively linked to each other in a manner as to provide the aforementioned data collection, preferably, in a suitable way for a query search.

The present invention further relates to a device for providing an indication of severity of a metabolic disease in a subject known or suspected to suffer from said metabolic disease, said device comprising an analysis unit and, operatively connected thereto, an evaluation unit, wherein (i) said analysis unit is configured to determine in a sample of said subject at least partial nucleic acid sequences of at least two alleles of a gene contributing to said metabolic disease or at least partial amino acid sequences of the polypeptides expressed therefrom; and wherein (ii) said evaluation unit comprises a, preferably tangibly embedded, evaluation algorithm comparing the nucleic acid or amino acid sequences determined by the analysis unit to values in a data collection as specified herein, and outputting a value of a residual enzyme activity corresponding to said sequences and/or an indication of severity of disease, preferably via an output unit.

The term “device”, as used herein, relates to a functional combination of the indicated means, i.e. in which the means are operatively linked to each other. Said means may be implemented in a single device or may be physically separated devices which are operatively linked to each other. The data storage medium, preferably, comprises the aforementioned data collection or database. The analysis unit may be any unit adapted to provide the sequence information as specified herein above. Thus, the system of the present invention allows identifying whether a test data set is comprised by the data collection stored in the data storage medium and, if yes, to output a value of a residual enzyme activity corresponding to said sequences and/or an indication of severity of disease. Consequently, the method for identifying a subject suffering from a severe form of a metabolic disease can be implemented by the system of the present invention.

The present invention also relates to a kit comprising at least two of (i) means for determining at least partial nucleic acid sequences of at least two alleles of a gene contributing to a metabolic disease or at least partial amino acid sequences of the polypeptides expressed therefrom; (ii) means for determining a residual activity of the polypeptides expressed from said at least two alleles of a gene contributing to said metabolic disease, and (iii) a data collection as specified herein.

The term “kit”, as used herein, refers to a collection of the aforementioned components. Preferably, said components are combined with additional components, preferably within an outer container. Examples for such components of the kit as well as methods for their use have been given in this specification. Preferably, the means for determining at least partial nucleic acid sequences is at least one sequencing and/or amplification primer. Also preferably, the means for determining a residual activity of the polypeptides is at least one expression vector. The kit, preferably, contains the aforementioned components in a ready-to-use formulation. The outer container, also preferably, comprises instructions for carrying out a method of the present invention. Additionally, such user's manual may provide instructions about correctly using the components of the kit. A user's manual may be provided in paper or electronic form, e.g., stored on CD or CD ROM. The present invention also relates to the use of said kit in any of the methods according to the present invention.

Furthermore, the present invention relates to a computer program product comprising instructions which, when executed on a suitable computer, cause at least the following steps to be performed

(i) obtaining at least partial nucleic acid sequences of at least two alleles of a gene contributing to a metabolic disease in a subject or at least partial amino acid sequences of the polypeptides expressed therefrom;

(ii) comparing said at least partial nucleic acid sequences or at said least partial amino acid sequences of (i) to a database comprising at least sequence data of least two alleles of said gene related to a metabolic disease and a value of a residual enzyme activity correlating therewith; and

(iii) providing an output indicating at least one of (I) the residual activity obtained from the database in step (ii), (II) an indication on the severity of said metabolic disease, and (III) recommendations on further treatment of said subject.

The invention further discloses and proposes a computer program including computer-executable instructions for performing the method according to the present invention in one or more of the embodiments enclosed herein when the program is executed on a computer or computer network. Specifically, the computer program may be stored on a computer-readable data carrier. Thus, specifically, one, more than one or even all of method steps as indicated above may be performed by using a computer or a computer network, preferably by using a computer program.

The invention further discloses and proposes a computer program product having program code means, in order to perform the method according to the present invention in one or more of the embodiments enclosed herein when the program is executed on a computer or computer network. Specifically, the program code means may be stored on a computer-readable data carrier. Further, the invention discloses and proposes a data carrier having a data structure stored thereon, which, after loading into a computer or computer network, such as into a working memory or main memory of the computer or computer network, may execute the method according to one or more of the embodiments disclosed herein.

The invention further proposes and discloses a computer program product with program code means stored on a machine-readable carrier, in order to perform the method according to one or more of the embodiments disclosed herein, when the program is executed on a computer or computer network. As used herein, a computer program product refers to the program as a tradable product. The product may generally exist in an arbitrary format, such as in a paper format, or on a computer-readable data carrier. Specifically, the computer program product may be distributed over a data network.

Finally, the invention proposes and discloses a modulated data signal which contains instructions readable by a computer system or computer network, for performing the method according to one or more of the embodiments disclosed herein.

Preferably, referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements.

Specifically, the present invention further discloses:
A computer or computer network comprising at least one processor, wherein the processor is adapted to perform the method according to one of the embodiments described in this description,
a computer loadable data structure that is adapted to perform the method according to one of the embodiments described in this description while the data structure is being executed on a computer,
a computer program, wherein the computer program is adapted to perform the method according to one of the embodiments described in this description while the program is being executed on a computer,
a computer program comprising program means for performing the method according to one of the embodiments described in this description while the computer program is being executed on a computer or on a computer network,
a computer program comprising program means according to the preceding embodiment, wherein the program means are stored on a storage medium readable to a computer,
a storage medium, wherein a data structure is stored on the storage medium and wherein the data structure is adapted to perform the method according to one of the embodiments described in this description after having been loaded into a main and/or working storage of a computer or of a computer network, and
a computer program product having program code means, wherein the program code means can be stored or are stored on a storage medium, for performing the method according to one of the embodiments described in this description, if the program code means are executed on a computer or on a computer network.

In view of the above, the following embodiments are particularly envisaged: Embodiment 1. A method for identifying a subject suffering from a severe form of a metabolic disease, comprising
a) obtaining (i) at least partial nucleic acid sequences of at least two alleles of a gene contributing to said metabolic disease or at least partial amino acid sequences of the polypeptides expressed therefrom; and (ii) a residual activity of the polypeptides expressed from said at least two alleles, preferably by simultaneous determination of the activity of the polypeptides expressed from the at least two alleles in a host cell or an extract thereof, and
b) based on the result of step a), identifying a subject suffering from a severe form of a metabolic disease.

Embodiment 2. The method of embodiment 1, wherein said obtaining at least partial nucleic acid sequences or at least partial amino acid sequences is determining in a sample of said subject at least partial nucleic acid sequences of at least two alleles of a gene contributing to said metabolic disease or at least partial amino acid sequences of the polypeptides expressed therefrom.

Embodiment 3. The method of embodiment 1 or 2, wherein said obtaining at least partial nucleic acid sequences of at least two alleles in step a) comprises sequencing the nucleic acid sequences of at least subsequences of said at least two alleles.

Embodiment 4. The method of any one of embodiments 1 to 3, wherein said obtaining at least partial nucleic acid sequences of at least two alleles in step a) comprises sequencing at least 25%, preferably at least 50%, more preferably at least 75%, most preferably all, of the nucleic acid sequences of cDNAs derived from said at least two alleles.

Embodiment 5. The method of any one of embodiments 1 to 4, wherein said determining the sequences of the polypeptides expressed from at least two alleles of a gene contributing to said metabolic disease comprises determining at least 25%, preferably at least 50%, more preferably at least 75%, most preferably 100%, of the amino acid sequences of said polypeptides.

Embodiment 6. The method of any one of embodiments 1 to 5, wherein said obtaining a residual activity is determining in a sample of said subject a residual activity of the polypeptides expressed from said at least two alleles.

Embodiment 7. The method of any one of embodiments 1 to 6, wherein step a) is determining in a sample of said subject (i) at least partial nucleic acid sequences of at least two alleles of a gene contributing to said metabolic disease or at least partial amino acid sequences of the polypeptides expressed therefrom; and/or (ii) a residual activity of the polypeptides expressed from said at least two alleles.

Embodiment 8. The method of any one of embodiments 1 to 7, wherein said step a) comprises determining the residual activity of the polypeptides expressed from said at least two alleles of a gene contributing to said metabolic disease.

Embodiment 9. The method of any one of embodiments 1 to 8, wherein said step a) comprises determining in a sample of said subject (i) at least partial nucleic acid sequences of at least two alleles of a gene contributing to said metabolic disease or at least partial amino acid sequences of the polypeptides expressed therefrom; and (ii) the residual activity of the polypeptides expressed from said at least two alleles of a gene contributing to said metabolic disease.

Embodiment 10. The method of any one of embodiments 1 to 9, wherein said step a) comprises determining at least partial nucleic acid sequences of at least two alleles of a gene contributing to said metabolic disease or at least partial amino acid sequences of the polypeptides expressed therefrom and retrieving corresponding values of a residual activity from a database.

Embodiment 11. The method of any one of embodiments 1 to 10, wherein said obtaining the residual activity of step a) comprises
(i) cloning the sequences of at least two alleles of step a) into expression constructs,
(ii) expressing said expression constructs, preferably in a suitable host cell, and
(iii) determining the residual activity in an enzymatic assay.

Embodiment 12. The method of embodiment 11, wherein said expression constructs comprise expression control sequences and wherein said expression control sequences of said expression constructs are essentially identical, preferably are identical, between the constructs for the at least two, preferably the two, alleles.

Embodiment 13. The method of embodiment 11 or 12, wherein expressing said expression constructs comprises contacting said host cell with similar, preferably identical, amounts of said expression constructs.

Embodiment 14. The method of any one of embodiments 11 to 13, wherein expressing said expression constructs further comprises contacting said host cell with an expression construct encoding a transfection control.

Embodiment 15. The method of any one of embodiments 1 to 14, wherein said determining the residual activity comprises normalizing the activity determined to the activity of a reference enzyme and/or to the amount of protein used.

Embodiment 16. The method of any one of embodiments 1 to 15, wherein said at least two alleles are two alleles.

Embodiment 17. The method of any one of embodiments 1 to 15, wherein said alleles are naturally occurring alleles.

Embodiment 18. The method of any one of embodiments 1 to 17, wherein said subject is a mammal, preferably a human.

Embodiment 19. The method of any one of embodiments 1 to 18, wherein said metabolic disease is a disease caused by an enzymatic activity of one type of polypeptide in body cells of said subject which is diminished relative to the enzymatic activity in corresponding body cells of a subject not affected by said metabolic disease.

Embodiment 20. The method of any one of embodiments 1 to 19, wherein said metabolic disease is a monogenic metabolic disease.

Embodiment 21. The method of any one of embodiments 1 to 20, wherein said metabolic disease is a recessive metabolic disease.

Embodiment 22. The method of any one of embodiments 1 to 21, wherein said metabolic disease is an autosomal metabolic disease.

Embodiment 23. The method of any one of embodiments 1 to 22, wherein said metabolic disease is an autosomal recessive metabolic disease.

Embodiment 24. The method of any one of embodiments 1 to 23, wherein said metabolic disease is a monogenic autosomal recessive metabolic disease.

Embodiment 25. The method of any one of embodiments 1 to 24, wherein said metabolic disease is Citrullinemia type 1 (CTLN1) or Argininosuccinate Lyase deficiency (ASL-D).

Embodiment 26. The method of any one of embodiments 1 to 25, wherein said metabolic disease is Citrullinemia type 1 (CTLN1).

Embodiment 27. The method of any one of embodiments 1 to 26, wherein said gene contributing to said metabolic disease is a mutated form of the argininosuccinate synthetase 1 (ASS1) gene.

Embodiment 28. The method of embodiment 27, wherein said ASS1 gene is the human ASS1 gene, preferably the gene with Genbank Acc No: NG_011542.1.

Embodiment 29. The method of any one of embodiments 1 to 25, wherein said metabolic disease is Argininosuccinate Lyase deficiency (ASL-D).

Embodiment 30. The method of embodiment 29, wherein said gene contributing to said metabolic disease is a mutated form of the argininosuccinate lyase (ASL) gene, preferably the gene with Genbank Acc No.: AF376770.1 Embodiment 31. A method of determining a residual activity of an enzyme expressed from at least two alleles of a gene related to a metabolic disease, comprising
A) expressing said two alleles in a host cell, and
B) determining the activity of said enzyme in said host cell or an extract thereof.

Embodiment 32. The method of embodiment 31, further comprising a feature of any one of embodiments 1 to 30.

Embodiment 33. A data collection comprising sequence data of least two alleles of a gene related to a metabolic disease and a value of a residual enzyme activity correlating therewith.

Embodiment 34. The data collection of embodiment 33 comprised on a data carrier.

Embodiment 35. A device for providing an indication of severity of a metabolic disease in a subject known or suspected to suffer from said metabolic disease, said device comprising an analysis unit and, operatively connected thereto, an evaluation unit, wherein (i) said analysis unit is configured to determine in a sample of said subject at least partial nucleic acid sequences of at least two alleles of a gene contributing to said metabolic disease or at least partial amino acid sequences of the polypeptides expressed therefrom; and wherein (ii) said evaluation unit comprises a, preferably tangibly embedded, evaluation algorithm comparing the nucleic acid or amino acid sequences determined by the analysis unit to values in a data collection according to embodiment 33 or 34, and outputting a value of a residual enzyme activity corresponding to said sequences and/or an indication of severity of disease.

Embodiment 36. A kit comprising at least two of (i) means for determining at least partial nucleic acid sequences of at least two alleles of a gene contributing to a metabolic disease or at least partial amino acid sequences of the polypeptides expressed therefrom; (ii) means for determining a residual activity of the polypeptides expressed from said at least two alleles of a gene contributing to said metabolic disease, and (iii) a data collection according to embodiment 33 or 34.

Embodiment 37. The kit of embodiment 36, wherein said means for determining at least partial nucleic acid sequences is at least one sequencing and/or amplification primer.

Embodiment 38. The kit of embodiment 36 or 37, wherein said means for determining a residual activity of the polypeptides is at least one expression vector.

Embodiment 39. A computer program product comprising instructions which, when executed on a suitable computer, cause at least the following steps to be performed
(i) obtaining at least partial nucleic acid sequences of at least two alleles of a gene contributing to a metabolic disease in a subject or at least partial amino acid sequences of the polypeptides expressed therefrom;
(ii) comparing said at least partial nucleic acid sequences or at said least partial amino acid sequences of (i) to a database comprising at least sequence data of least two alleles of said gene related to a metabolic disease and a value of a residual enzyme activity correlating therewith; and
(iii) providing an output indicating at least one of (I) the residual activity obtained from the database in step (ii), (II) an indication on the severity of said metabolic disease, and (III) recommendations on further treatment of said subject.

Embodiment 40. A method of identifying a compound for treating and/or preventing a metabolic disease, comprising

a) contacting a host cell expressing at least two alleles of a gene contributing to said metabolic disease with a candidate compound,

b) simultaneously determining residual activity of the polypeptides expressed from said at least two alleles in said host cell or an extract thereof,

c) comparing the residual activity determined in step b) to a control activity, and

d) based on comparison step c), identifying a compound for treating and/or preventing said metabolic disease.

Embodiment 41. The method of embodiment 40, further comprising a feature of any one of embodiments 1 to 30.

All references cited in this specification are herewith incorporated by reference with respect to their entire disclosure content and the disclosure content specifically mentioned in this specification.

### FIGURE LEGENDS

FIG. 1: Residual enzymatic ASS1 activity correlates with initial biochemical parameters. (A) NH₄⁺ₘₐₓ (μmol/l) subject to residual enzymatic ASS1 activity (%) as determined in the biallelic expression system. Each point represents a single patient (n=52). Grey line displays estimated regression curve. GAM-analysis, p<0.001, R=0.30. (B) Boxplot illustrating NH₄⁺ₘₐₓ (μmol/l) with a residual enzymatic ASS1 activity below or equal to 8.1% (n=32) and above 8.1% (n=20). Data are shown as median (black thick line) and mean (triangle), length of the box corresponds to interquartile range (IQR), upper and lower whiskers correspond to max. 1.5×IQR, each point represents an outlier. Recursive partitioning, p<0.001. (C) Peak plasma L-citrulline concentration subject to residual enzymatic ASS1 activity (%). Each point represents a single patient (n=33). Grey line displays estimated regression curve. GAM-analysis, p<0.001, R=0.46. ASS1, argininosuccinate synthetase 1.

FIG. 2: Residual enzymatic ASS1 activity predicts number and severity of HAEs.

(A) Number of HAEs (NH₄⁺ₘₐₓ≥100 μmol/l) per year subject to residual enzymatic ASS1 activity (%). Each point represents a single patient (n=43). Grey line displays estimated regression curve. GAM-analysis, p=0.002, R=0.26. (B) Boxplot illustrating number of HAEs (NH₄⁺ₘₐₓ≥100 μmol/l) per year with a residual enzymatic ASS1 activity below or equal to 8.1% (n=17) or above 8.1% (n=26). Data are shown as median (black thick line) and mean (triangle), length of the box corresponds to IQR, upper and lower whiskers correspond to max. 1.5×IQR, each point represents an outlier. Recursive partitioning, p=0.003. (C) NH₄⁺ₘₐₓ during severest HAE subject to residual enzymatic ASS1 activity (%). Each point represents a single patient (n=26). Grey line displays estimated regression curve. GAM-analysis, p=0.007, R=0.23. (D) Boxplot illustrating NH₄⁺ₘₐₓ during severest HAE with a residual enzymatic ASS1 activity below or equal to 8.1% (n=17) or above 8.1% (n=9). Data are shown as median (black thick line) and mean (triangle), length of the box corresponds to IQR, upper and lower whiskers correspond to max. 1.5×IQR, each point represents an outlier. Recursive partitioning, p=0.01. ASS1, argininosuccinate synthetase 1.

FIG. 3: Residual enzymatic ASS1 activity predicts neurocognitive outcome. (A) Cognitive SDS subject to residual enzymatic ASS1 activity (%). Each point represents a single patient (n=34). Grey line indicates estimated regression curve. GAM analysis, p=0.003. (B) Boxplot illustrating cognitive SDS with a residual enzymatic ASS1 activity below or equal to 8.1% (n=13) and above 8.1% (n=21). Data are shown as median (black thick line) and mean (triangle), length of the box corresponds to IQR, upper and lower whiskers correspond to max. 1.5×IQR. Recursive partitioning, p=0.031. (C) Levelplot for cognitive SDS, residual enzymatic ASS1 activity and age at testing (years). Cognitive SDS values are indicated by color coding in grading from light gray to black with descending cognitive SDS. ASS1, argininosuccinate synthetase 1.

FIG. 4: Residual enzymatic ASS1 activity predicts organ-specific manifestations. (A) Boxplot illustrating occurrence of movement disorders (%) for individuals with a residual enzymatic ASS1 activity below or equal to 19.3% (n=34) and above 19.3% (n=26). Grey shading corresponds to individuals with movement disorders in each group. Recursive partitioning, p=0.03. (B) Boxplots displaying occurrence of hepatocellular injury (%) for individuals with a residual enzymatic ASS1 activity below or equal to 3.9% (n=7) and above 3.9% (n=48). Grey shading corresponds to individuals with episode(s) of hepatocellular injury in each group. Recursive partitioning, p=0.039. ASS1, argininosuccinate synthetase 1.

FIG. 5: Decision for liver transplantation and special education reflects risk-stratification by residual enzymatic ASS1 activity. (A) Boxplot illustrating proportion of individuals (%) with a liver graft for those with a residual enzymatic ASS1 activity below or equal to 4.8% (n=13) and above 4.8% (n=58). Grey shading corresponds to individuals with liver graft in each group. Recursive partitioning, p=0.011. (B) Boxplots displaying proportion of individuals (%) with special education for those with a residual enzymatic ASS1 activity below or equal to 26.6% (n=26) and above 26.6% (n=12). Grey shading corresponds to individuals with special education in each group. Recursive partitioning, p<0.001. ASS1, argininosuccinate synthetase 1.

FIG. 6: Overview of relative mRNA expression levels per variant combination. COS-7 cells were transfected with 2.5 μg of each FLAG- and MYC-tagged ASS1 expression vectors and 1 μg of beta-galactosidase reporter plasmid, cultured for 48 hours and subjected to quantitative analysis of mRNA expression applying qRT-PCR. Data are expressed as fold-change (mean+/−SD) normalized to the relative expression of the respective ASS1 wildtype plasmids (A-E; n=3 for each experiment). Gray columns illustrate FLAG-tagged expression vectors, black columns represent MYC-tagged plasmids.

FIG. 7: Overview of protein expression levels per variant combination. COS-7 cells were transfected with 2.5 μg of each FLAG- and MYC-tagged ASS1 expression vectors and 1 μg of beta-galactosidase reporter plasmid, cultured for 48 hours and subjected to protein expression analysis using standard Western blot technique (A-E). Expression of FLAG- or MYC-tagged ASS1 variants was visualized using anti-FLAG- or anti-MYC antibodies. Equal protein expression levels in cell lysates were confirmed by immunoblotting using an anti-β-actin antibody.

FIG. 8: Overview of residual enzymatic ASS1 activities per variant combination. COS-7 cells were transfected with 2.5 μg of each FLAG- and MYC-tagged ASS1 expression vectors and 1 μg of beta-galactosidase reporter plasmid, cultured for 48 hours and residual enzymatic ASS1 activities determined as described under Material and methods. Data are expressed as mean+/−SD in % of ASS1 wildtype activity (A-E, n=3 for each experiment).

FIG. 9: (A) Peak plasma L-citrulline concentration reflects residual enzymatic ASS1 activity (%). Boxplot illustrating residual enzymatic ASS1 activity (%) with peak plasma L-citrulline concentration below or equal to 1335 μmol/l (n=18) and above 1335 μmol/l (n=15). Data are shown as median (black thick line) and mean (triangle), length of the box corresponds to IQR, upper and lower whiskers correspond to max. 1.5×IQR. Recursive partitioning, p<0.001. ASS1, argininosuccinate synthetase 1. (B) Correlation between disease onset (EO, LO, Asymptomatic) and residual enzymatic ASS1 activity (%). Boxplot illustrating correlation between disease onset (EO, LO, Asymptomatic) and residual enzymatic ASS1 activity (n=69). Individuals with EO (n=41; p<0.001) and LO (n=5; p=0.002) have lower mean residual enzymatic ASS1 activity than asymptomatic individuals (n=23). Mean residual enzymatic ASS1 activity between EO and LO individuals does not differ (p=0.19), ANOVA. Data are shown as median (black thick line) and mean (triangle), length of the box corresponds to IQR, upper and lower whiskers correspond to max. 1.5×IQR, each point represents an outlier. ASS1, argininosuccinate synthetase 1; EO, early onset; LO, late onset.

FIG. 10: Enzymatic ASL activity correlates with initial NH₄⁺ₘₐₓ as well as the annual frequency and severity of HAEs. (A) NH₄⁺ₘₐₓ (μmol/l) subject to enzymatic ASL activity as determined in the mammalian biallelic expression system. Each point represents a single patient (n=46). Gray line displays estimated regression curve. GAM analysis, p<0.001, R²=0.36). (B) Boxplot illustrating NH₄⁺ₘₐₓ (μmol/L) with an enzymatic activity below or equal to 7.9% (n=17) and above 7.9% (n=29). Data are shown as median (black thick line) and mean (triangle), length of the box corresponds to the interquartile range (IQR), upper and lower whiskers correspond to max. 1.5×IQR, each point represents an outlier. Recursive partitioning, p<0.001. (C) Number of HAEs (NH₄⁺ₘₐₓ≥100 μmol/l) per year subject to enzymatic ASL activity (%). Each point represents a single patient (n=35). Gray line displays estimated regression curve. GAM analysis, p<0.001, R2=0.39. (D) NH₄⁺ₘₐₓ during most severe HAE subject to enzymatic ASL activity (%). Each point represents a single patient (n=19). Gray line displays the estimated regression curve. GAM analysis, p=0.019, R²=0.32. (E) Boxplot illustrating number of HAEs (NH₄⁺ₘₐₓ≥100 μmol/l) per year with an enzymatic ASS1 activity below or equal to 8.7% (n=10) or above 8.7% (n=25). Data are shown as median (black thick line) and mean (triangle), length of the box corresponds to IQR, upper and lower whiskers correspond to max. 1.5×IQR, each point represents an outlier. Recursive partitioning, p=0.004. ASL, argininosuccinate lyase; HAEs, hyperammonemic events (defined as NH4+>100 μmol/l).

The following Examples shall merely illustrate the invention. They shall not be construed, whatsoever, to limit the scope of the invention.

### EXAMPLE 1: MATERIALS AND METHODS

### Eligibility Criteria

Only individuals from the UCDC and E-IMD patient registries with confirmed ASS1-D and unequivocal molecular genetic test results of ASS1 on the cDNA-level were included into this analysis. A detailed description of the data model of both registries, information on written informed consent as well as the follow-up protocols used were described in detail recently (Posset et al. (2019), J Inherit Metab Dis. 42(1):93-106; Burgard et al. (2016), J Inherit Metab Dis. 39(2):219-29). Requirements set forth by the ICMJE (International Committee of Medical Journal Editors) are met. All procedures followed were in accordance with the ethical standards of the Helsinki Declaration of 1975, as revised in 2013. Data were retrieved from the UCDC and E-IMD electronic databases with cut-off for data pull on 10 Oct. 2018.

### Plasmids

To generate the tagged wildtype ASS1 expression vectors, the ASS1 coding sequence was amplified by PCR with MYC- or FLAG-tags introduced at the C- or N-terminus using specific primer pairs and inserted into the BamHI and NotI restriction sites in the open-reading frame of the eukaryotic expression vector pcDNA5 (Thermo Fisher Scientific). Pathogenic ASS1 gene variants derived from individuals with CTLN1 within the E-IMD and UCDC registries were introduced into the tagged ASS1 expression vectors using the QuickChange II site-directed mutagenesis kit (Agilent) according to the manufacturer's suggestions. The wildtype ASS1 expression vectors and the correct insertion of the mutations were confirmed by Sanger-sequencing. The pSV-β-galactosidase control vector (Promega) was kindly provided by N. Himmelreich (Heidelberg University, Germany).

### Cell Culture and Transfections

COS-7 cells were maintained as adherent cell culture in 10 cm petri-dishes in DMEM medium (Thermo Fisher Scientific) supplemented with 10% heat-inactivated Fetal Bovine Serum in a humified incubator at 37° C. and 5% CO₂. COS-7 cells were transfected with 2.5 μg of each FLAG- and MYC-tagged ASS1-plasmid and 1 μg of beta-galactosidase reporter plasmid using Lipofectamin 2000 reagent (Thermo Fisher Scientific) according to the manufacturer's instructions. After 48 hours, cells were washed two times with ice-cold phosphate-buffered saline (PBS), lysed and the lysates subjected to subsequent downstream applications (e.g. qRT-PCR, Western blotting or ASS1 enzyme assay).

qRT-PCR

RNA was extracted by standard procedure with TRIzol reagent (Invitrogen). Equal amounts from each sample (500 to 800 ng) were used for cDNA syntheses applying the Maxima first strand cDNA synthesis kit (Thermo Fisher Scientific). Real-time qPCR was performed on a CFX Connect™ 180 Real-Time cycler (Biorad) (denaturation step: 95° C. for 25 s, annealing and elongation step: 60° C. for 30 s) using SensiFast SYBR™ Hi-ROX mix (Bioline) with the following primers:

Tagged-ASS1-plasmids (N-terminus):
N-FLAG_forward:
(SEQ ID NO: 1)
5′-CTACAAAGACGATGACGACAAG-3′
N-MYC_forward:
(SEQ ID NO: 2)
5′-GAAGAGGATCTGGGAGGTTCAGG-3′
N-tag_reverse:
(SEQ ID NO: 3)
3′-CTTCTTCCTGGCTTCCTCG-5′
Tagged-ASS1-plasmids (C-terminus):
C-tag_forward:
(SEQ ID NO: 4)
5′-CCCACTGTCTCTCTACAATGAGG-3′
C-FLAG_reverse:
(SEQ ID NO: 5)
3′-GTCGTCATCGTCTTTGTAGTCG-5′
C_MYC_reverse:
(SEQ ID NO: 6)
3′-GTTTTTGTTCGCTGCCTCCTG-5′
β-Actin:
Actin_forward:
(SEQ ID NO: 7)
5′-CAACCTTCCTTCCTGGGCAT-3′
Actin-reverse:
(SEQ ID NO: 8)
3′-GATTTTCATCGTGCTGGGCG-3′

The expression level of beta-actin was used for normalization.

### Western Blot

48 hours after transfection, COS-7 cells were washed two times in ice-cold PBS and lysed in 1× ice-cold radioimmunoprecipitation buffer (600 mM NaCl, 100 mM TRIS-HCl pH 7.4, 10 mM EDTA, 2% Triton X-100, 0.2% SDS, 1% sodium deoxycholate) followed by additional sonification. Hereafter, lysates were centrifuged at 13,000×g and 4° C. for 10 minutes, and supernatants were used for Western blotting according to standard laboratory protocols. For protein visualization, membranes were probed with the following primary antibodies: anti-FLAG (1:2,000, BioLegend), anti-MYC (1:2,000, Cell Signaling) and anti-3-Actin (1:2,000, Sigma-Aldrich).

### Spectrophotometric Analysis of the ASS1 Enzyme

Purified citrate synthase, malate dehydrogenase, fumarase from porcine heart were purchased from Sigma-Aldrich. Recombinant C-terminally FLAG-tagged argininosuccinate lyase protein was obtained from Creative Biomart. ASS1 enzyme activity was determined in transfected COS-7 cell lysates (triplicates) in a buffer containing 10 mmol/l potassium phosphate, 10 mmol/l TRIS-HCl, 2 mmol/l citrulline, 600 mU/ml of argininosuccinate lyase, 650 mU/ml fumarase, 660 mU/ml malate dehydrogenase, 400 mU/ml citrate synthase, 2 mmol/l aspartic acid, 330 μmol/l NAD and 2 mmol/l ATP, which was adjusted to pH 7.4 (25° C.). ASS1 enzyme activity was determined as NAD reduction at a λ=340-400 nm. Lysates of COS-7 transfected with empty pcDNA5 vector served as negative control. Negative control values were subtracted from ASS1 activities to adjust for unspecific ASS1 background activity.

To control for transfection efficacy, the ASS1 activities were normalized to the beta-galactosidase activity in each sample as determined by the β-galactosidase enzyme assay system (Promega) according to the manufacturer's instructions. The adjusted ASS1 activities were normalized to the protein content in the respective samples. Residual activities are depicted as percentage of total (%) by dividing the normalized ASS1 activity of a specific mutational combination (homozygous/compound heterozygous) by the normalized wildtype ASS1 activities.

### Clinical Variables Used for Data Analyses

The following variables were used for data analyses: disease onset (EO, LO, asymptomatic), NH₄⁺ₘₐₓ, peak plasma L-citrulline concentration, movement disorder (dystonia and/or chorea and/or ataxia), tone change (muscular hypotonia and/or muscular hypertonia and/or spasticity), cognitive SDS at last regular visit being calculated using the normative data from the standardization sample of each cognitive test, special education (self-contained class as well as any supportive services), hepatocellular injury (alanine aminotransferase ≥250 U/l or aspartate aminotransferase ≥250 U/l), liver transplantation (LTx), kidney function [full age spectrum glomerular filtration rate (FAS-GFR)≥90 ml/min/1.73 m² versus FAS-GFR<90 ml/min/1.73 m²], number of HAEs (with NH₄⁺ₘₐₓ>100 μmol/l) per year of observation (defined as time between date of birth and last regular visit), NH₄⁺ₘₐₓ during severest hyperammonemic decompensation.

For symptomatic individuals with reported HAE during the initial presentation/decompensation, biochemical data (NH₄⁺ₘₐₓ and peak plasma L-citrulline concentration) represent the highest value prior to initiation of treatment. For symptomatic CTLN1-individuals without reported HAE during initial presentation, NH₄⁺ₘₐₓ was defined as upper limit of normal range (50 μmol/l). For asymptomatic CTLN1-individuals, NH₄⁺ₘₐₓ and peak plasma L-citrulline concentrations represent highest reported follow-up values during the observation period as conservative approach. Only untreated asymptomatic CTLN1-individuals were considered for analysis.

We investigated the impact of the cumulative residual ASS1 enzymatic activities as determined by the established biallelic expression system on clinical outcome parameters outlined above. Mortality could not be examined due to a low number of deceased individuals within the observation period.

### Statistical Analysis

All analyses were performed using R (www.r-project.org). To evaluate the relationship between a continuous dependent variable and the cumulative residual enzymatic ASS1 activity as predictor variable, a generalized additive regression model (GAM) with automated smoothing selections was used (Wood (2011) J R Stat Soc B. 73:3-36). In GAM, the linear relationship between the response variable and predictors are replaced by nonlinear smooth functions. The R package “mgcv” was used to fit GAM regressions. We used unbiased recursive partitioning to determine cut-off values for the impact of cumulative residual enzymatic ASS1 activity on outcome variables (Hothorn et al. (2006) J Comput Graph Stat 15:651-74). Two groups where compared with a t-test with Welch correction. P values reported were two-sided. P≤0.05 was considered statistically significant.

### EXAMPLE 2: RESULTS

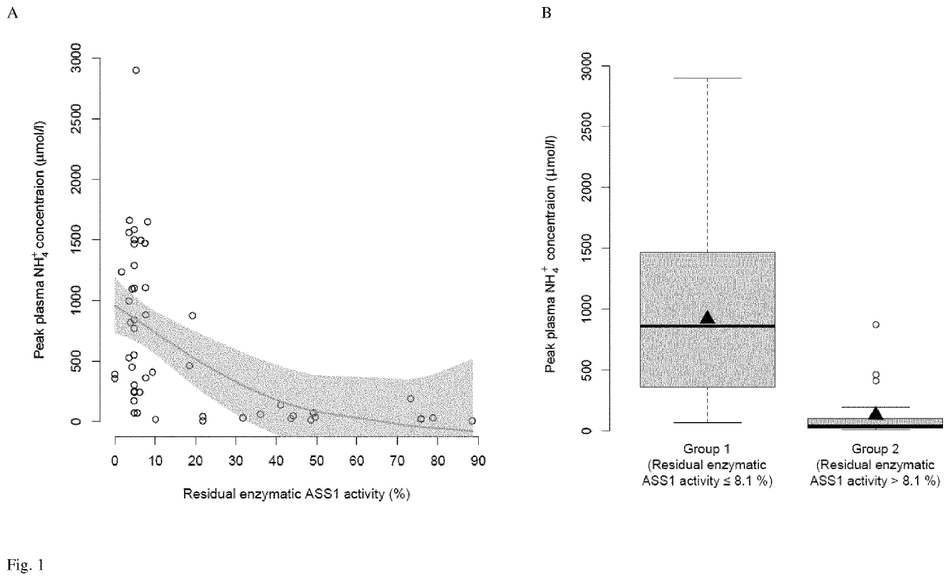

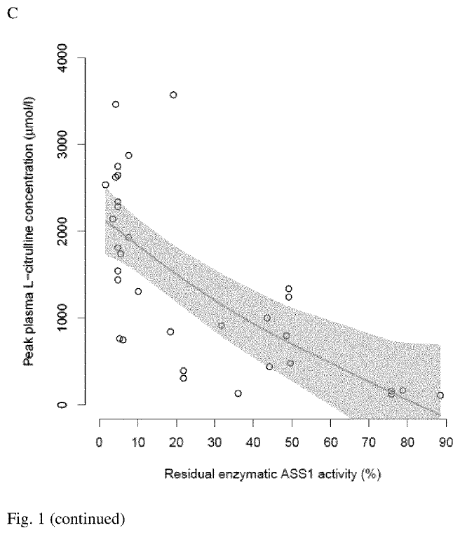

Residual enzymatic ASS1 activity correlates with initial biochemical alterations Since enzymatic ASS1 dysfunction results in hyperammonemia and hypercitrullinemia, we first studied whether NH₄⁺ₘₐₓ and L-citrulline concentrations at initial decompensation are associated with residual enzymatic ASS1 activity and hence reflect disease severity. Notably, initial NH₄⁺ₘₐₓ inversely correlated with the underlying residual enzymatic ASS1 activity (n=52, p<0.001, R²=0.30, GAM-analysis; FIG. 1a) as determined in the mammalian biallelic expression system. Furthermore, we identified a threshold distribution for individuals with CTLN1 with a residual enzymatic ASS1 activity below or equal to 8.1% exhibiting significantly higher NH₄⁺ₘₐₓ at initial decompensation as opposed to patients with a residual enzymatic ASS1 activity above to 8.1% (n=52, p<0.001, recursive partitioning; FIG. 1b). Additionally, also peak plasma L-citrulline concentrations showed an inverse correlation with the underlying residual enzymatic ASS1 activity (n=33, p<0.001, R²=0.46, GAM-analysis; FIG. 1c).

### Residual Enzymatic ASS1 Activity Predicts the Clinical Disease Course and Neurocognitive Outcome

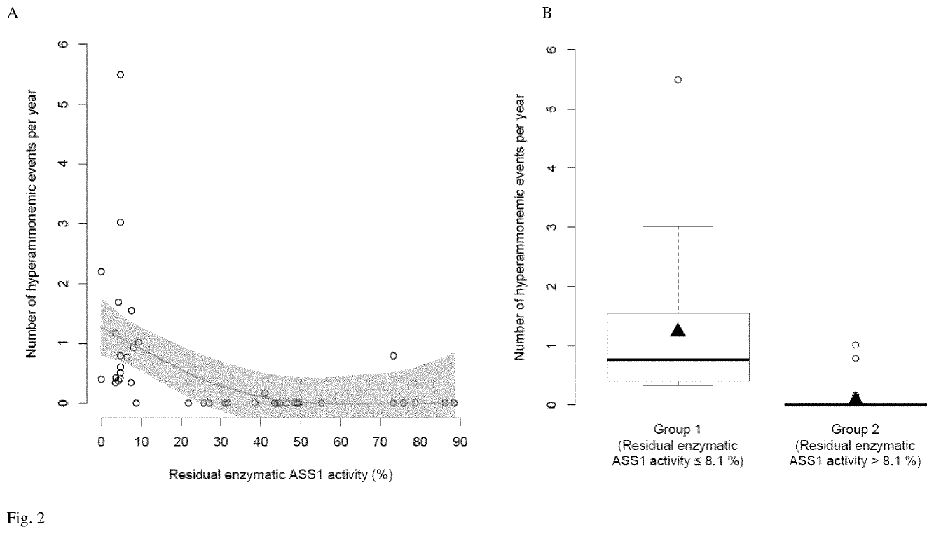

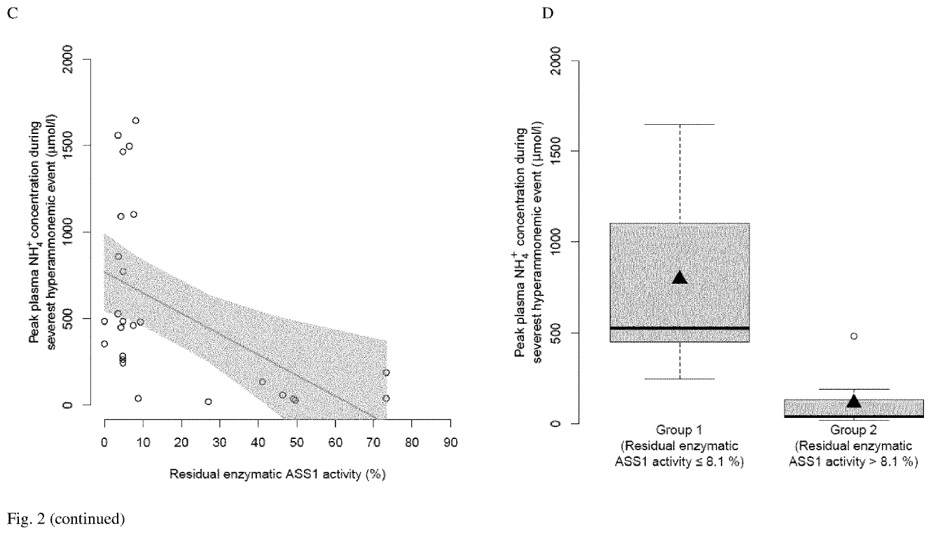

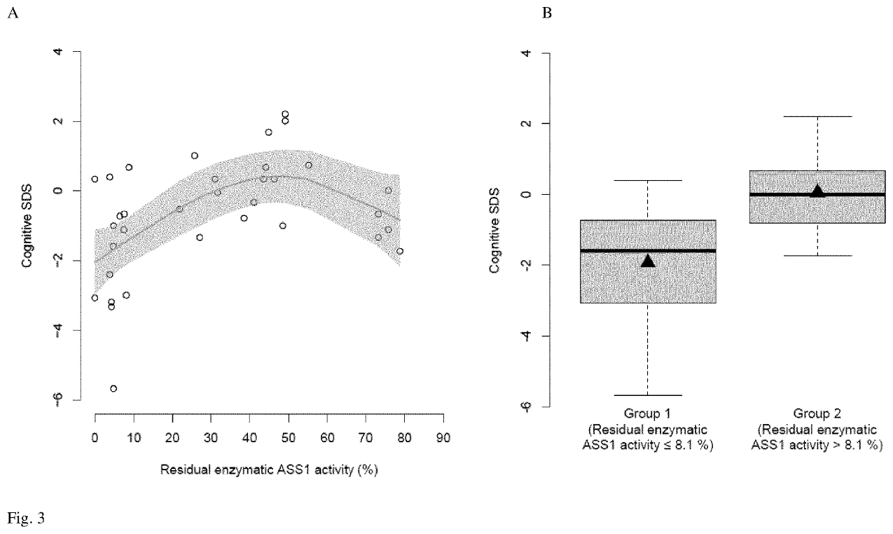

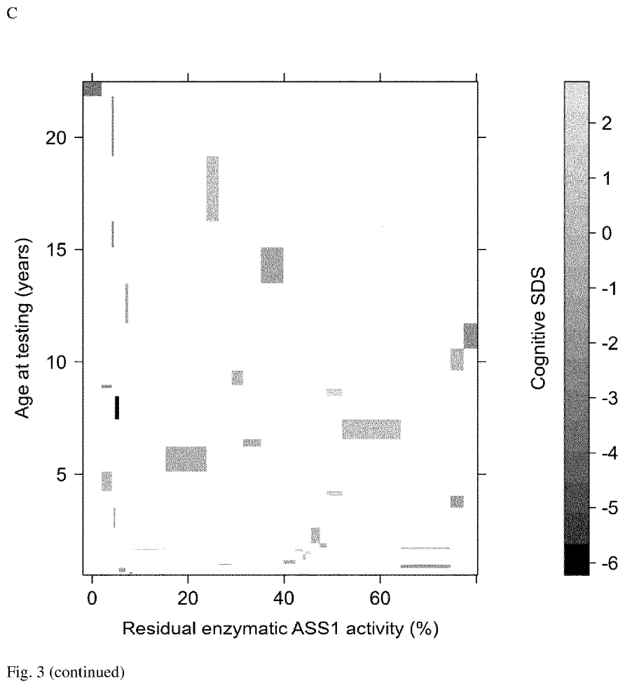

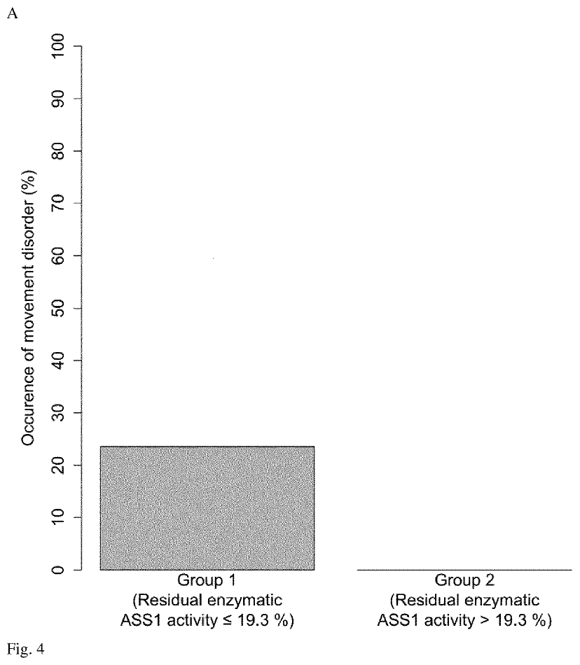

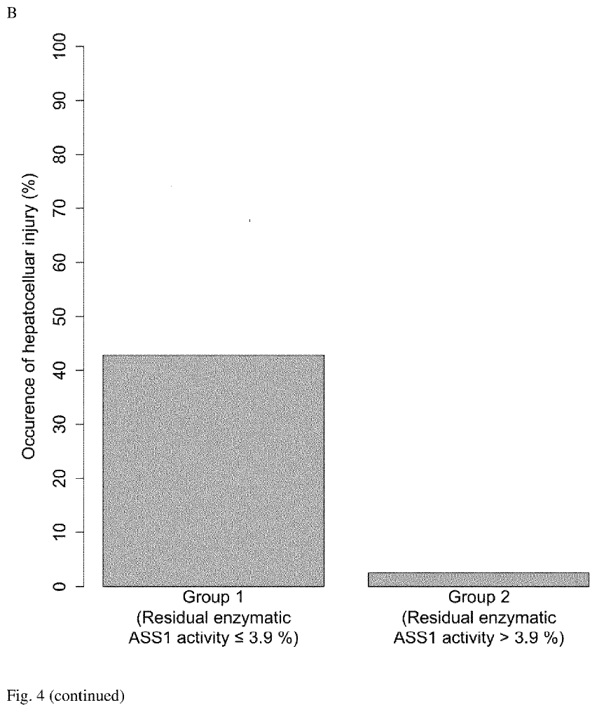

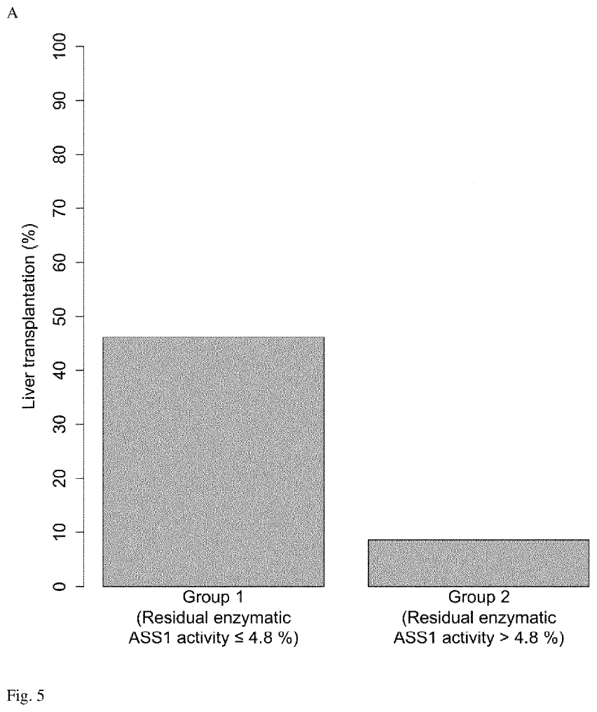

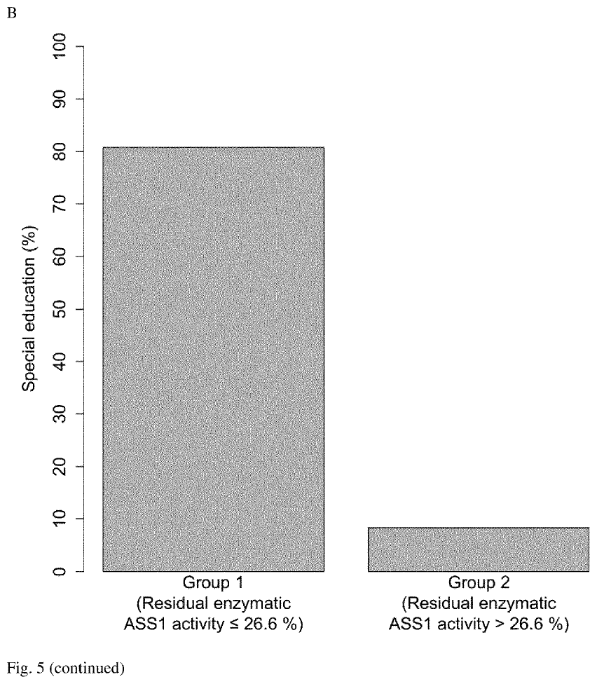

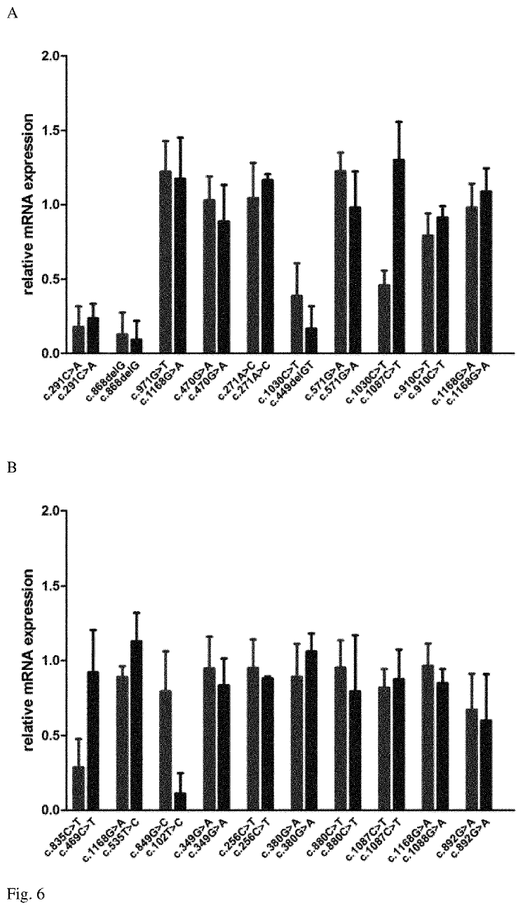

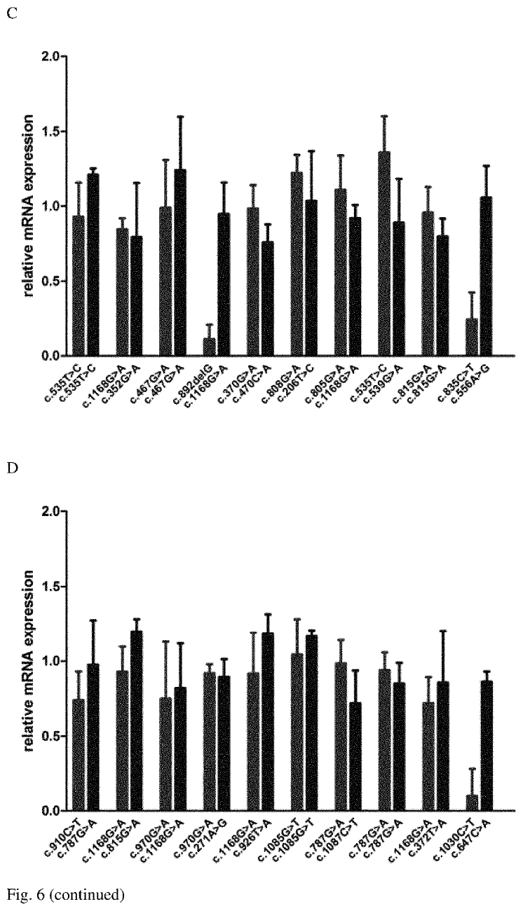

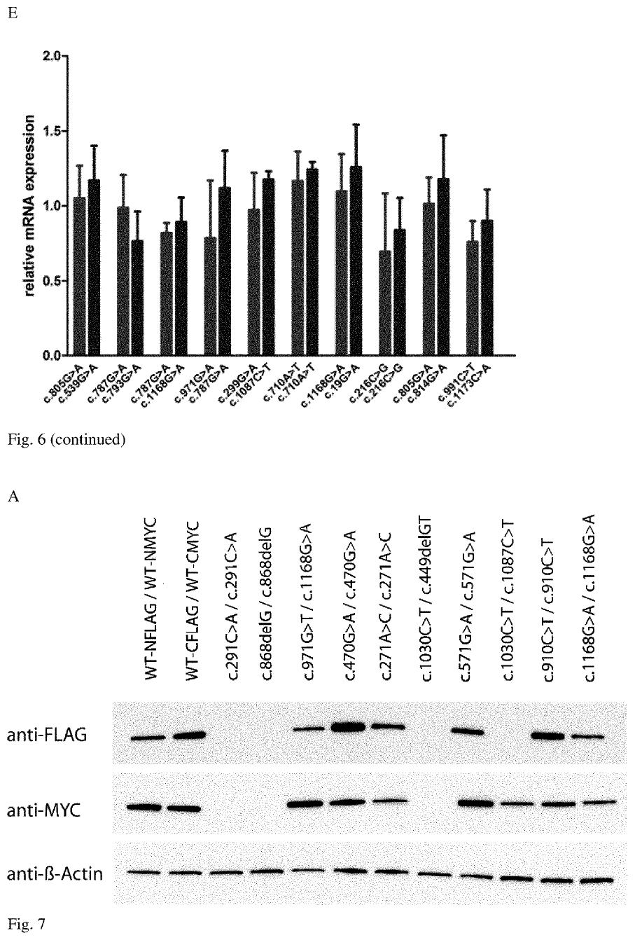

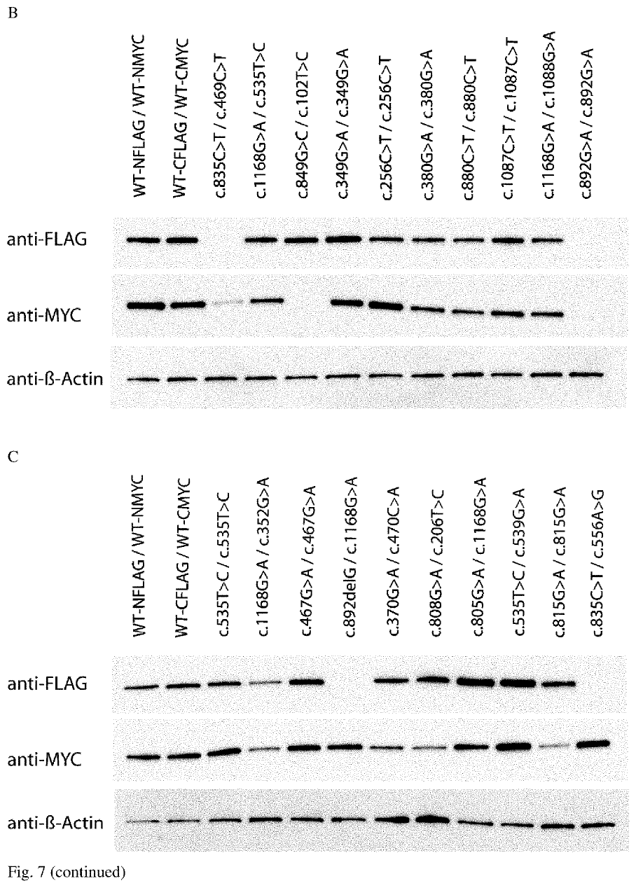

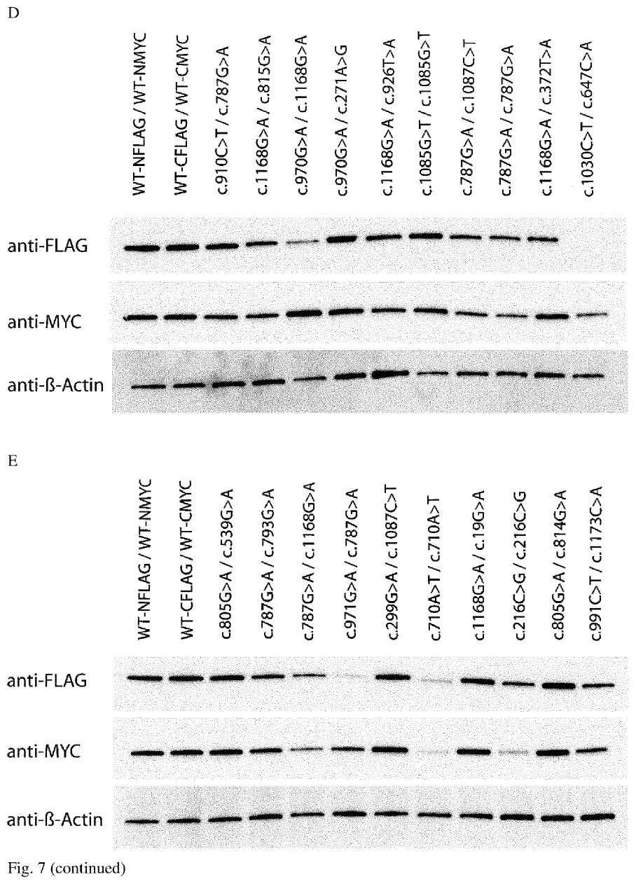

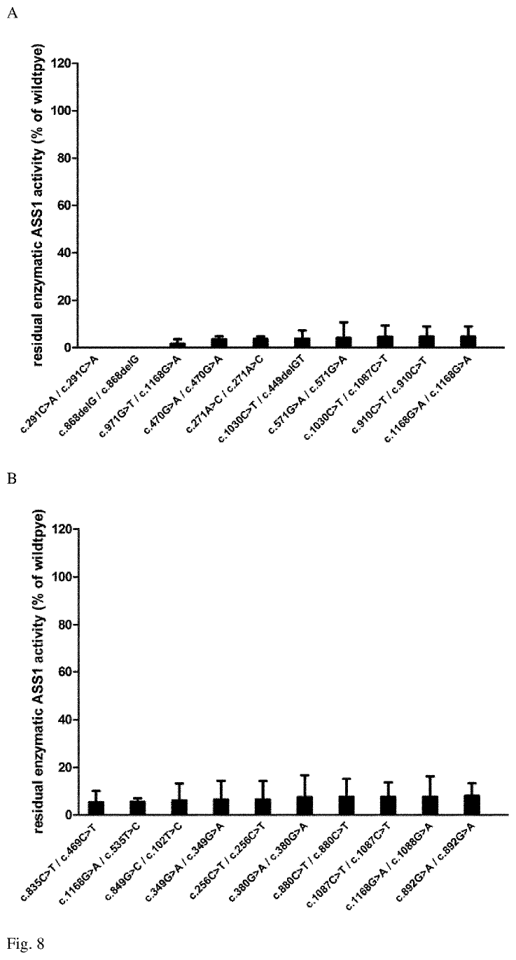

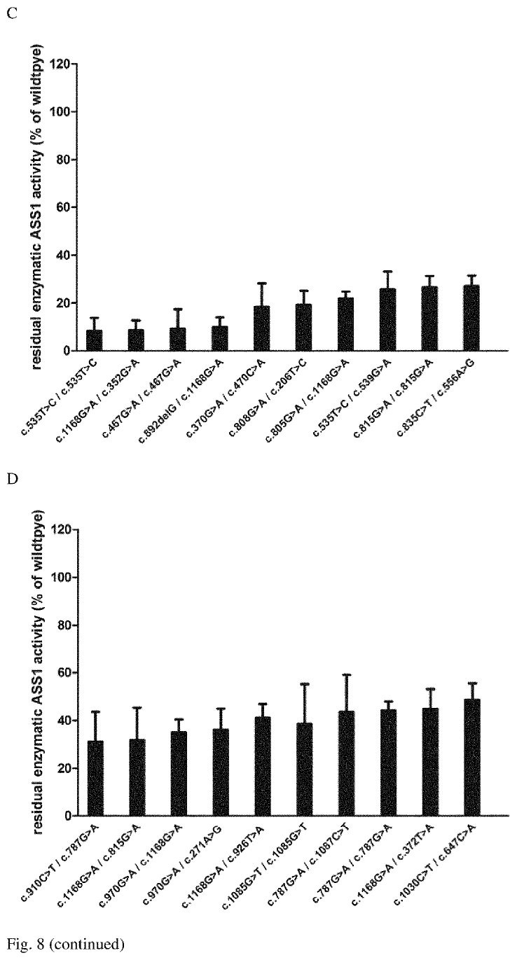

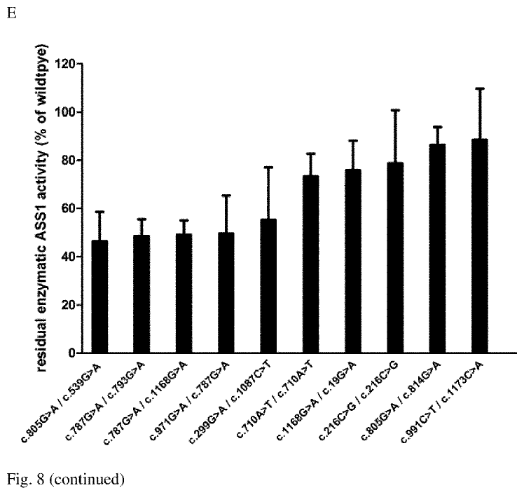

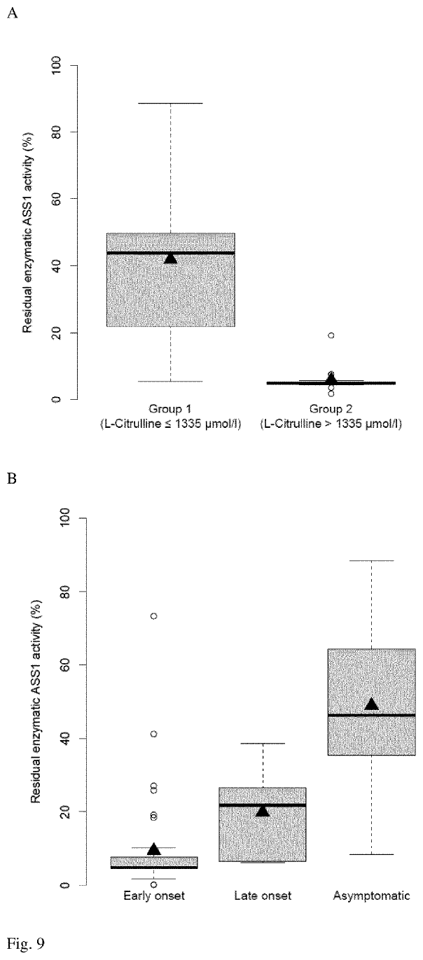

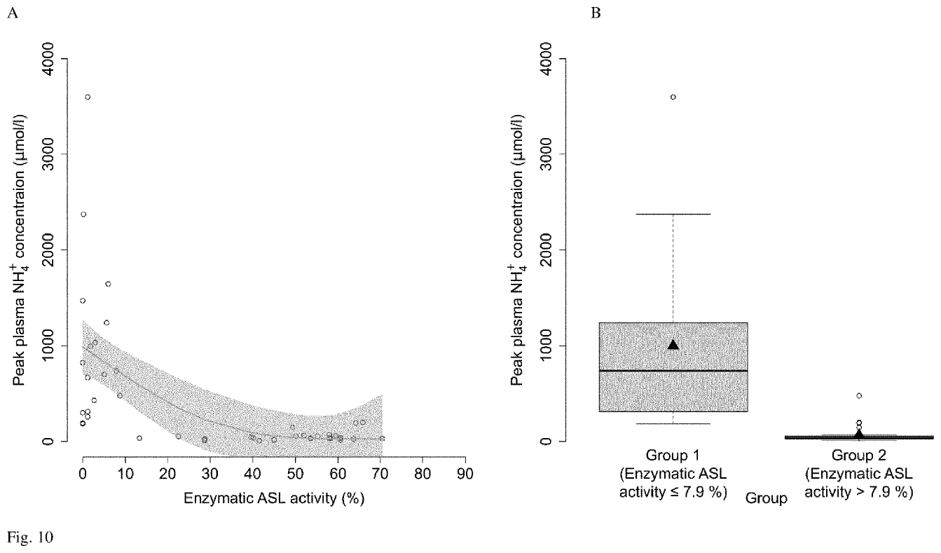

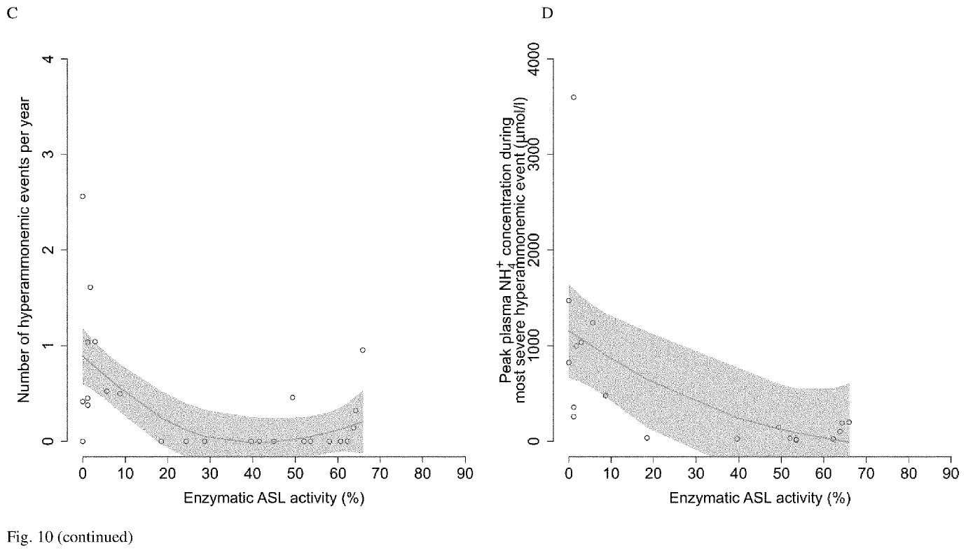

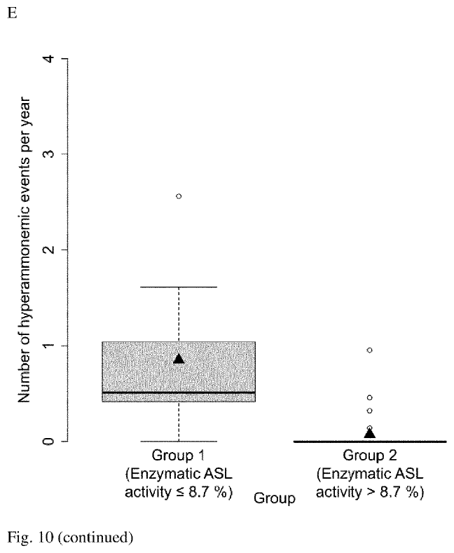

Subsequently, we assessed whether residual enzymatic ASS1 activities correlate with the clinical disease course as indicated by reported number of HAE per year, NH₄⁺ₘₐₓ during severest hyperammonemic decompensation and the cognitive SDS of individuals with CTLN1 at last regular visit. Individuals with a residual enzymatic ASS1 activity below or equal to 8.1% not only had a significantly higher number of HAE per year (n=43, p=0.003, recursive partitioning) and higher NH₄⁺ₘₐₓ during severest hyperammonemic decompensation (n=26, p=0.01, recursive partitioning) (FIG. 2a-d), but also exhibit lower neurocognitive function as opposed to individuals above this threshold (n=34, p=0.031, recursive partitioning) (FIG. 3 a, b). Intriguingly, besides residual enzyme activity also age seems to affect cognitive functions since older individuals with CTLN1 show lower cSDS than younger individuals (FIG. 3c).

Residual Enzymatic ASS1 Activity is Associated with Neurologic and Hepatic Outcome Parameters

Given the above described correlations of residual enzymatic ASS1 activity with the metabolic disease course and neurocognitive outcome, we next investigated, whether this observation also holds true for other clinical outcome parameters. Indeed, residual enzymatic ASS1 activity is associated with the appearance of movement disorders and hepatocellular injury. CTLN1-individuals with a residual enzymatic ASS1 activity below or equal to 19.3% exhibited more often movement disorders (n=60, p=0.03, recursive partitioning) (FIG. 4a). This observation could be corroborated for the appearance of hepatocellular injury with a residual enzymatic ASS1 activity of 3.9% as threshold (n=55, p=0.039, recursive partitioning) (FIG. 4b). However, residual enzymatic ASS1 activity did not discriminate between individuals with or without tone changes (n=65, p=0.052, Asymptomatic Wilcoxon-Mann-Whitney U-test) neither between CTLN1 individuals with (FAS-GFR<90 ml/min/1.73 m²) or without (FAS-GFR≥90 ml/min/1.73 m²) reported episodes of impaired kidney function (n=47, p=0.47, t-test).

### Empiric Clinical Practice Already Reflects a Potential Future Risk-Stratification Based on Residual Enzymatic ASS1 Activity

Since residual enzymatic ASS1 activity predicts the disease course and clinical outcome in CTLN1, we wondered whether current clinical practice as indicated by LTx status and implementation of special education in the management already reflect a future potential risk-stratification based on residual enzymatic ASS1 activities. Within our study population, individuals with a residual enzymatic ASS1 activity below or equal to 4.8% underwent LTx more often (n=71, p=0.011, recursive partitioning) (FIG. 5a) and importantly, CTLN1 individuals with a residual enzymatic ASS1 activity above 10% never underwent LTx. Intriguingly, residual enzymatic ASS1 activity also mirrors the presence of special education in the management. CTLN1 individuals with a residual enzymatic ASS1 activity below or equal to 26.6% received special education support more frequently (n=38, p<0.001, recursive partitioning) than above this threshold (FIG. 5b).

### EXAMPLE 3: DESCRIPTION OF STUDY POPULATION AND ASS1 PROTEIN FUNCTIONS

We determined enzymatic ASS1 activities in a group of 71 CTLN1-individuals, representing 48 ASS1 gene variants, and 50 different combinations in total. Results of qRT-PCR and Western Blot analysis of ASS1 mRNAs and proteins are depicted in FIGS. 6 and 7. Residual enzymatic ASS1 activities per variant combination are illustrated in FIG. 8. Detailed descriptive characteristics of the study population are shown in Tables 1 and 2 below. Correlation between peak plasma L-citrulline concentration and residual enzymatic ASS1 activity and correlation between disease onset and residual enzymatic ASS1 activity are shown in FIG. 9.

TABLE 1
Descriptive characteristics for correlation analyses in CTLN 1 - part I
Correlation between residual ASS1 activity and peak plasma NH₄⁺ concentration

| | | Peak plasma NH₄⁺ | Peak plasma NH₄⁺ |
|---|---|---|---|
| ASS1 activity | Peak plasma NH₄⁺ | concentration (μmol/l), | concentration (μmol/l), |
| (%) | concentration (μmol/l) | if ASS1 activity ≤ 8.1% | if ASS1 activity > 8.1% |
| Mean, SD | Mean, SD | Mean, SD | Mean, SD |
| Median [Q1, Q3] | Median [Q1, Q3] | Median [Q1, Q3] | Median [Q1, Q3] |
| Min, Max; n | Min, Max; n | Min, Max; n | Min, Max; n |
| 20.0, 24.4 | 613.7, 645.5 | 917.4, 639.7 | 127.8, 216.5 |
| 6.3 [4.8, 32.9] | 373.9 [66.7, 1093.5] | 859.5 [358.8, 1464.7] | 37.0 [21.5, 85.9] |
| 0, 88.5; 52 | 9, 2900; 52 | 69.3, 2900; 32 | 9.0, 872.0; 20 |

Correlation between residual ASS1 activity and peak plasma L-citrulline concentration

| | | Peak plasma L-citrulline | Peak plasma L-citrulline |
|---|---|---|---|
| ASS1 activity | Peak plasma L-citrulline | concentration (μmol/l), | concentration (μmol/l), |
| (%) | concentration (μmol/l) | if ASS1 acitivity ≤ 19.3% | if ASS1 acitivity > 19.3% |
| Mean, SD | Mean, SD | Mean, SD | Mean, SD |
| Median [Q1, Q3] | Median [Q1, Q3] | Median [Q1, Q3] | Median [Q1, Q3] |
| Min, Max; n | Min, Max; n | Min, Max; n | Min, Max; n |
| 25.5, 26.3 | 1422.4, 1030.2 | 2078.6, 839.7 | 542.6, 434.3 |
| 10.1 [4.8, 44.1] | 1305.0 [478.0, 2283.0] | 2140.0 [1490.0, 2636.5] | 416.5 [160.0, 886.0] |
| 1.7, 88.5; 33 | 108.0, 3574.0; 33 | 751.0, 3574.0; 19 | 108.0, 1335.0; 14 |

Correlation between residual ASS1 activity and number of hyperammonemic
events (HAE) per year

| | | Number of HAE | Number of HAE |
|---|---|---|---|
| ASS1 activity | Number of HAE | per year, if ASS1 | per year, if ASS1 |
| (%) | per year | acitivity ≤ 8.1% | acitivity > 8.1% |
| Mean, SD | Mean, SD | Mean, SD | Mean, SD |
| Median [Q1, Q3] | Median [Q1, Q3] | Median [Q1, Q3] | Median [Q1, Q3] |
| Min, Max; n | Min, Max; n | Min, Max; n | Min, Max; n |
| 31.1, 27.7 | 0.53, 1.02 | 1.23, 1.33 | 0.08, 0.25 |
| 27.1 [4.8, 48.8] | 0 [0, 0.68] | 0.76 [0.42, 1.54] | 0 [0, 0] |
| 0, 88.5; 43 | 0, 5.5; 43 | 0.34, 5.5; 17 | 0, 1.0; 26 |

Correlation between residual ASS1 activity and peak plasma NH₄⁺ concentration during
severest HAE

| | | Peak plasma NH₄⁺ | Peak plasma NH₄⁺ |
|---|---|---|---|
| ASS1 activity | Peak plasma NH₄⁺ | concentration (μmol/l), | concentration (μmol/l), |
| (%) | concentration (μmol/l) | if ASS1 activity ≤ 8.1% | if ASS1 activity > 8.1% |
| Mean, SD | Mean, SD | Mean, SD | Mean, SD |
| Median [Q1, Q3] | Median [Q1, Q3] | Median [Q1, Q3] | Median [Q1, Q3] |
| Min, Max; n | Min, Max; n | Min, Max; n | Min, Max; n |
| 17.5, 22.7 | 560.4, 524.8 | 796.4, 498.3 | 114.7, 149.1 |
| 5.6 [4.4, 22.7] | 455.0 [148.3, 837.5] | 526.0 [450.0, 1103.0] | 40.0 [37.0, 134.0] |
| 0, 73.3; 26 | 19.0, 1647.0; 26 | 245.44, 1647.0; 17 | 19.0, 482.0; 9 |

Correlation between residual ASS1 activity and cognitive SDS at last regular visit

| ASS1 activity | | Cognitive SDS, | Cognitive SDS, |
|---|---|---|---|
| (%) | Cognitive SDS | if ASS1 activity ≤ 8.1% | if ASS1 activity > 8.1% |
| Mean, SD | Mean, SD | Mean, SD | Mean, SD |
| Median [Q1, Q3] | Median [Q1, Q3] | Median [Q1, Q3] | Median [Q1, Q3] |
| Min, Max; n | Min, Max; n | Min, Max; n | Min, Max; n |
| 30.7, 25.9 | −0.7, 1.7 | −1.9, 1.7 | 0.1, 1.1 |
| 29.1 [5.2, 48.0] | −0.7 [−1.3, 0.3] | −1.6 [−3.1, −0.7] | 0 [−0.8, 0.7] |
| 0, 78.8; 34 | −5.7, 2.2; 34 | −5.7, 0.4; 13 | −1.7, 2.2; 21 |

Descriptive characteristics for the correlation between residual enzymatic ASS1 activity and peak plasma NH4⁺ concentration, peak plasma L-citrulline concentration, number of HAE per year, peak plasma NH4⁺ concentration during severest HAE, and cSDS at last regular visit, respectively. Of note, cut-off values of residual enzymatic ASS1 activity for various outcome variables, i.e. peak plasma NH₄⁺ concentration, number and severity of HAE and cSDS, correspond to 8.1%.
ASS1, argininosuccinate synthetase 1, cSDS, cognitive SDS value , HAE, hyperammonemic events defined as NH4⁺ > 100 μmol/l.

TABLE 2
Descriptive characteristics for correlation analyses in CTLN1 - part II
Correlation between residual ASS1 activity and occurrence of movement disorder (MD)

| ASS1 activity | ASS1 acitivity | | |
|---|---|---|---|
| of individuals | of individuals | | |
| with MD (%) | w/o MD (%) | | |
| Mean, SD | Mean, SD | MD, if ASS1 | MD, if ASS1 |
| Median [Q1, Q3] | Median [Q1, Q3] | activity ≤ 19.3% | activity > 19.3% |
| Min, Max; n | Min, Max; n | (% of total) | (% of total) |
| 6.7, 5.6 | 26.4, 24.5 | 8/34 = 23.5% | 0/26 = 0% |
| 4.8 [4.8, 7.7] | 20.2 [5.5, 44.3] | | |
| 0, 19.2; 8 | 1.7, 88.5; 52 | | |

Correlation between residual ASS1 activity and occurrence of hepatocellular injury (HCI)

| ASS1 activity | ASS1 acitivity | | |
|---|---|---|---|
| of individuals | of individuals | | |
| with HCI (%) | w/o HCI (%) | | |
| Mean, SD | Mean SD | HCI, if ASS1 | HCI, if ASS1 |
| Median [Q1, Q3] | Median [Q1, Q3] | activity ≤ 3.9% | activity > 3.9% |
| Min, Max; n | Min, Max; n | (% of total) | (% of total) |
| 3.2, 3.3 | 28.9, 26.7 | 3/7 = 42.9% | 2/48 = 4.2% |
| 3.84 [0, 4.8] | 21.9 [4.9, 46.0] | | |
| 0, 7.6; 5 | 1.7, 88.5; 50 | | |

Correlation between residual ASS1 activity and liver transplantation (LTx) status

| ASS1 activity | ASS1 acitivity | | |
|---|---|---|---|
| of individuals | of individuals | | |
| with LTx (%) | w/o LTx (%) | | |
| Mean, SD | Mean, SD | LTx, if ASS1 | LTx, if ASS1 |
| Median [Q1, Q3] | Median [Q1, Q3] | activity ≤ 4.8% | activity > 4.8% |
| Min, Max; n | Min, Max; n | (% of total) | (% of total) |
| 5.4, 2.1 | 26.0, 25.5 | 6/13 = 46.2% | 5/58 = 8.6% |
| 4.7 [3.5, 7.0] | 14.3 [4.8, 44.3] | | |
| 3.5, 9.3; 11 | 0, 88.5; 60 | | |

Correlation between residual ASS1 activity and special education

| ASS1 activity | ASS1 acitivity | | |
|---|---|---|---|
| of individuals | of individuals | | |
| with special | w/o special | | |
| education (%) | education (%) | Special education, | Special education, |
| Mean, SD | Mean, SD | if ASS1 | if ASS1 |
| Median [Q1, Q3] | Median [Q1, Q3] | activity ≤ 26.6% | activity > 26.6% |
| Min, Max; n | Min, Max; n | (% of total) | (% of total) |
| 10.4, 16.3 | 35.1, 22.8 | 21/26 = 80.8% | 1/12 = 8.3% |
| 4.8 [4.7, 7.9] | 35.2 [21.0, 49.1] | | |
| 0, 78.8; 22 | 3.9, 75.8; 16 | | |

Descriptive characteristics for the correlation between residual enzymatic ASS1 activity and MD, HCI, LTx status and special education, respectively. Individual cut-off values could be identified for all outcome variables. Of note, all patients receiving LTx had residual enzymatic ASS1 activity below 10%.
HCI, hepatocellular injury defined as occurrence of alanine aminotransferase and or aspartate aminotransferase ≥ 250 U/l, LTx, liver transplant status, MD, movement disorder defined as occurrence of dystonia and/or chorea and/or ataxia, special education defined as self-contained class as well as any supportive services.

### EXAMPLE 4: ARGININOSUCCINIC ACIDURIA (ASA)

Only individuals from the UCDC and E-IMD patient registries with confirmed ASA and unequivocal molecular genetic test results of ASL were included in this analysis. If not otherwise indicated, methods were essentially as described above, with the necessary adaptations to ASA.

It was first investigated whether biochemical parameters at initial decompensation are associated with the degree of enzymatic dysfunction and hence reflect disease severity also in ASA. Initial NH₄⁺ₘₐₓ inversely correlated with the underlying enzymatic ASL activity (FIG. 10A) as determined in the mammalian biallelic expression system. Also, there is a threshold distribution for ASA with an enzymatic ASL activity below or equal to 7.900 leading to higher initial NH₄⁺ₘₐₓ as opposed to individuals with an enzymatic ASL activity above this threshold (n=46, FIG. 10B). Next, it was assessed whether enzymatic ASL activity correlates and therefore might predict disease severity as reflected by the reported number of HAEs/year of observation, NH₄⁺ₘₐₓ during the most severe HAE and the cognitive SDS of individuals with ASA at last follow-up visit. Enzymatic ASL activity was not only associated with the number of HAEs/year (n=35; FIG. 10C), but also with NH₄⁺ₘₐₓ during the most severe hyperammonemic decompensation (n=19, FIG. 10D). Individuals with an enzymatic ASL activity below or equal to 8.7% showed a significantly higher number of HAEs/year as opposed to individuals with an ASL activity above this threshold (n=35; FIG. 10E).

Non-standard literature cited:
Burgard et al. (2016), J Inherit Metab Dis. 39(2):219-29.
Diez-Fernandez et al. (2017), Hum Mutat. 38(5):471-84.
Hothorn et al. (2006) J Comput Graph Stat 15:651-74.
Kolker et al. (2015), J Inherit Metab Dis.; 38(6):1041-57.
Kolker et al. (2015) J Inherit Metab Dis. 38(6):1059-74.
Summar et al. (2013), Mol Genet Metab 110(1-2):179-80.
Posset et al. (2019), J Inherit Metab Dis. 42(1):93-106.
Posset et al. (2019) Ann Neurol. doi: 10.1002/ana.25492, PMID: 31018246.
Posset et al. (2016), J Inherit Metab Dis. 39(5):661-72.
Waisbren et al. (2018) J Inherit Metab Dis. 41(4):657-67.
Wood (2011) J R Stat Soc B. 73:3-36.

1. A method for identifying a subject suffering from a severe form of a metabolic disease, comprising
a) obtaining (i) at least partial nucleic acid sequences of at least two alleles of a gene contributing to said metabolic disease or at least partial amino acid sequences of the polypeptides expressed therefrom; and (ii) a residual activity of the polypeptides expressed from said at least two alleles by simultaneous determination of the activity of the polypeptides expressed from the at least two alleles in a host cell or an extract thereof, and
b) based on the result of step a), identifying a subject suffering from a severe form of a metabolic disease.

2. The method of claim 1, wherein step a) is determining in a sample of said subject (i) at least partial nucleic acid sequences of at least two alleles of a gene contributing to said metabolic disease or at least partial amino acid sequences of the polypeptides expressed therefrom; and/or (ii) a residual activity of the polypeptides expressed from said at least two alleles.

3. The method of claim 1, wherein said obtaining at least partial nucleic acid sequences of at least two alleles in step a) comprises sequencing the nucleic acid sequences of at least subsequences of said at least two alleles.

4. The method of claim 1, wherein said step a) comprises determining the residual activity of the polypeptides expressed from said at least two alleles of a gene contributing to said metabolic disease.

5. The method of claim 1, wherein said obtaining the residual activity of step a) comprises
(i) cloning the sequences of at least two alleles of step a) into expression constructs,
(ii) expressing said expression constructs, preferably in a suitable host cell, and
(iii) determining the residual activity in an enzymatic assay.

6. The method of claim 1, wherein said at least two alleles are two alleles.

7. The method of claim 1, wherein said alleles are naturally occurring alleles.

8. The method of claim 1, wherein said subject is a mammal.

9. The method of claim 1, wherein said metabolic disease is a disease caused by an enzymatic activity of one type of polypeptide in body cells of said subject which is diminished relative to the enzymatic activity in corresponding body cells of a subject not affected by said metabolic disease.

10. The method of claim 1, wherein said metabolic disease is Citrullinemia type 1 (CTLN1) or Argininosuccinate Lyase deficiency (ASL-D).

11. A method of determining a residual activity of an enzyme expressed from at least two alleles of a gene related to a metabolic disease, comprising
A) expressing said two alleles in a host cell, and
B) determining the activity of said enzyme in said host cell or an extract thereof, wherein said determining is simultaneous determination of the activity of the polypeptides expressed from the at least two alleles.

12. A data collection comprising sequence data of least two alleles of a gene related to a metabolic disease and a value of a residual enzyme activity correlating therewith.

13. (canceled)

14. (canceled)

15. (canceled)

16. (canceled)

17. The method of claim 1, wherein the subject is a human.

18. The method of claim 1, wherein the metabolic disease is a monogenic metabolic disease.

19. The method of claim 1, wherein the metabolic disease is a monogenic recessive metabolic disease.

20. The method of claim 1, wherein the metabolic disease is a monogenic autosomal recessive metabolic disease.

21. The data collection of claim 12, wherein the data collection is comprised on a data carrier.
